(19) United States
(12) Patent Application Publication
(10) Pub. No.: US 2024/0158507 A1
(43) Pub. Date: May 16, 2024

(54) VISTA Antagonist and Methods of Use

(71) Applicants: THE TRUSTEES OF DARTMOUTH COLLEGE, HANOVER, NH (US); KING'S COLLEGE LONDON, LONDON (GB)

(72) Inventors: Randolph J. Noelle, Plainfield, NH (US); Li Wang, Norwich, VT (US); Thomas Broughton, Lebanon, NH (US); Lorenzo F. Sempere, Grand Rapids, MI (US); Janet Louise Lines, Wooton-under-edge (GB)

(21) Appl. No.: 18/514,189

(22) Filed: Nov. 20, 2023

Related U.S. Application Data
(63) Continuation of application No. 17/092,817, filed on Nov. 9, 2020, now abandoned.
Application No. 17/092,817 is a continuation of application No. 14/686,422, filed on Apr. 14, 2015, now abandoned.
Application No. 14/686,422 is a continuation-in-part of application No. 14/534,793, filed on Nov. 6, 2014, now abandoned.
Application No. 14/534,793 is a continuation-in-part of application No. 13/925,094, filed on Jun. 24, 2013, now Pat. No. 10,745,467.
Application No. 13/925,094 is a continuation-in-part of application No. 13/637,381, filed on Apr. 2, 2013, now Pat. No. 9,631,018.
Application No. 13/637,381 is a continuation of application No. 12/732,371, filed on Mar. 26, 2010, now Pat. No. 8,231,872.
(60) Provisional application No. 61/979,219, filed on Apr. 14, 2014.
(60) Provisional application No. 61/663,969, filed on Jun. 25, 2012.
(60) Provisional application No. 61/663,431, filed on Jun. 22, 2012.

(51) Int. Cl. C07K 16/28; A61K 39/00; A61K 39/395; A61K 45/06; A61P 35/00; A61P 37/00; C07K 7/08; C07K 16/30

(52) U.S. Cl. CPC C07K 16/2827; A61K 39/00; A61K 39/0005; A61K 39/3955; A61K 45/06; A61P 35/00; A61P 37/00; C07K 7/08; C07K 16/30; C07K 16/3015; C07K 16/3023; A61K 38/00; A61K 2039/507; A61K 2039/57

(57) ABSTRACT

The present invention is directed to synergic or additive therapies comprising the administration of a VISTA antagonist and a PD-1, PD-L1 or POD-L3 antagonist; or the combination of a VISTA agonist and a -1, PD-L1 or POD-L3 agonist which combinations respectively elicit an additive or synergistic effect at promoting T cell immunity or inhibiting T cell immunity, i.e., CD4, CD8 or Th1 immunity. The agonists and antagonists may be in the same or separate compositions and may be administered together or separately administered in either order.

### CROSS-REFERENCE TO RELATED APPLICATIONS

This application is a continuation of U.S. Ser. No. 17/092,817 filed Nov. 9, 2020, which is a continuation of U.S. Ser. No. 14/686,422 filed Apr. 14, 2015, which claims priority to U.S. Provisional Ser. No. 61/979,219, filed Apr. 14, 2014, and is a continuation-in-part of U.S. Ser. No. 14/534,793, filed Nov. 6, 2014, which is a continuation-in-part of U.S. Ser. No. 13/925,094, filed Jun. 24, 2013, now U.S. Pat. No. 10,745,467, which claims priority to U.S. Provisional Ser. No. 61/663,969, filed Jun. 25, 2012, and U.S. Provisional Ser. No. 61/663,431, filed Jun. 22, 2012, and is a continuation-in-part of U.S. Ser. No. 13/637,381, filed Mar. 25, 2011, now U.S. Pat. No. 9,631,018, which is a U.S. 371 National Stage Entry of PCT/US2011/030087, filed Mar. 25, 2011, which is a continuation of U.S. Ser. No. 12/732,371, filed Mar. 26, 2010, now U.S. Pat. No. 8,231,872. The contents of each of the Provisional Ser. No. 61/979,219, U.S. Ser. No. 14/534,793, U.S. Ser. No. 13/925,094, U.S. Provisional Ser. No. 61/663,969, and U.S. Provisional Ser. No. 61/663,431 applications listed above, including the sequence listings, are incorporated herein by reference in their entireties.

### SEQUENCE LISTING DISCLOSURE

The contents of the electronic sequence listing (11432600001403.xml; Size: 2,280 bytes; and Date of Creation: Nov. 20, 2023) is herein incorporated by reference in its entirety.

### BACKGROUND

The immune system is tightly controlled by co-stimulatory and co-inhibitory ligands and receptors. These molecules provide not only a second signal for T cell activation but also a balanced network of positive and negative signals to maximize immune responses against infection while limiting immunity to self.

Induction of an immune response requires T cell expansion, differentiation, contraction and establishment of T cell memory. T cells must encounter antigen presenting cells (APCs) and communicate via T cell receptor (TCR)/major histocompatibility complex (MHC) interactions on APCs. Once the TCR/MHC interaction is established, other sets of receptor-ligand contacts between the T cell and the APC are required, i.e. co-stimulation via CD154/CD40 and CD28/B7.1-B7.2. The synergy between these contacts results in a productive immune response capable of clearing pathogens and tumors, and may be capable of inducing autoimmunity.

Another level of control has been identified, namely regulatory T cells (T_{reg}). This specific subset of T cells is generated in the thymus, delivered into the periphery, and is capable of constant and inducible control of T cells responses. Sakaguchi (2000) Cell 101(5):455-8; Shevach (2000) Annu. Rev. Immunol. 18:423-49; Bluestone and Abbas (2003) Nat. Rev. Immunol. 3(3):253-7. T_{reg} are represented by a CD4+CD25+ phenotype and also express high levels of cytotoxic T lymphocyte-associated antigen-4 (CTLA-4), OX-40, 4-1BB and the glucocorticoid inducible TNF receptor-associated protein (GITR). McHugh, et al. (2002) Immunity 16(2):311-23; Shimizu, et al. (2002) Nat. Immun. 3 (2):135-42. Elimination of T_{reg} cells by 5 day neonatal thymectomy or antibody depletion using anti-CD25, results in the induction of autoimmune pathology and exacerbation of T cells responses to foreign and self-antigens, including heightened anti-tumor responses. Sakaguchi, et al. (1985) J. Exp. Med. 161(1):72-87; Sakaguchi, et al. (1995) J. Immunol. 155(3):1151-64; Jones, et al. (2002) Cancer Immun. 2:1. In addition, T_{reg} have also been involved in the induction and maintenance of transplantation tolerance, since depletion of T_{reg} with anti-CD25 monoclonal antibodies results in ablation of transplantation tolerance and rapid graft rejection. Jarvinen, et al. (2003) Transplantation 76:1375-9. Among the receptors expressed by T_{reg} GITR seems to be an important component since ligation of GITR on the surface of Treg with an agonistic monoclonal antibody results in rapid termination of T_{reg} activity, resulting in autoimmune pathology and ablation of transplantation tolerance.

Costimulatory and co-inhibitory ligands and receptors not only provide a “second signal” for T cell activation, but also a balanced network of positive and negative signal to maximize immune responses against infection while limiting immunity to self. The best characterized costimulatory ligands are B7.1 and B7.2, which are expressed by professional APCs, and whose receptors are CD28 and CTLA-4. Greenwald, et al. (2005) Annu Rev Immunol 23, 515-548; Sharpe and Freeman (2002) Nat Rev Immunol 2, 116-126. CD28 is expressed by naïve and activated T cells and is critical for optimal T cell activation. In contrast, CTLA-4 is induced upon T cell activation and inhibits T cell activation by binding to B7.1/B7.2, thus impairing CD28-mediated costimulation. CTLA-4 also transduces negative signaling through its cytoplasmic ITIM motif. Teft, et al. (2006). Annu Rev Immunol 24, 65-97. B7.1/B7.2 KO mice are impaired in adaptive immune response (Borriello, et al. (1997) Immunity 6, 303-313; Freeman, et al. (1993) Science 262, 907-909), whereas CTLA-4 KO mice can not adequately control inflammation and develop systemic autoimmune diseases. Chambers, et al. (1997) Immunity 7, 885-895; Tivol, et al. (1995) Immunity 3, 541-547; Waterhouse, et al. (1995) Science 270, 985-988. The B7 family ligands have expanded to include costimulatory B7-H2 (ICOS Ligand) and B7-H3, as well as co-inhibitory B7-H1 (PD-L1), B7-DC (PD-L2), B7-H4 (B7S1 or B7x), and B7-H6. See Brandt, et al. (2009) J Exp Med 206, 1495-1503; Greenwald, et al. (2005) Annu Rev Immunol 23: 515-548.

Inducible costimulatory (ICOS) molecule is expressed on activated T cells and binds to B7-H2. See Yoshinaga, et al. (1999) Nature 402, 827-832. ICOS is important for T cell activation, differentiation and function, as well as essential for T-helper-cell-induced B cell activation, Ig class switching, and germinal center (GC) formation. Dong, et al. (2001) Nature 409, 97-101; Tafuri, et al. (2001) Nature 409, 105-109; Yoshinaga, et al. (1999) Nature 402, 827-832. Programmed Death 1 (PD-1) on the other hand, negatively regulates T cell responses. PD-1 KO mice develop lupus-like autoimmune disease, or autoimmune dilated cardiomyopathy depending upon the genetic background. Nishimura, et al. (1999) Immunity 11, 141-151. Nishimura, et al. (2001) Science 291: 319-322. The autoimmunity most likely results from the loss of signaling by both ligands PD-L1 and PD-L2. Recently, CD80 was identified as a second receptor for PD-L1 that transduces inhibitory signals into T cells. Butte, et al. (2007) Immunity 27: 111-122. The receptor for B7-H3 and B7-H4 still remain unknown.

The best characterized co-stimulatory ligands are B7.1 and B7.2, which belong to the Ig superfamily and are expressed on professional APCs and whose receptors are CD28 and CTLA-4 (Greenwald, et al. (2005) Annu. Rev. Immunol. 23:515-548). CD28 is expressed by naïve and activated T cells and is critical for optimal T cell activation. In contrast, CTLA-4 is induced upon T cell activation and inhibits T cell activation by binding to B7.1/B7.2, impairing CD28-mediated co-stimulation. B7.1 and B7.2 KO mice are impaired in adaptive immune response (Borriello, et al. (1997) Immunity 6:303-313), whereas CTLA-4 knockout mice cannot adequately control inflammation and develop systemic autoimmune diseases (Tivol, et al. (1995) Immunity 3:541-547; Waterhouse, et al. (1995) Science 270:985-988; Chambers, et al. (1997) Immunity 7:885-895).

The B7 family ligands have expanded to include co-stimulatory B7-H2 (inducible T cell co-stimulator (ICOS) ligand) and B7-H3, as well as co-inhibitory B7-H1 (PD-L1), B7-DC (PD-L2), B7-H4 (B7S1 or B7x), and B7-H6 (Greenwald, et al. (2005) supra; Brandt, et al. (2009) J. Exp. Med. 206:1495-1503). Accordingly, additional CD28 family receptors have been identified. ICOS is expressed on activated T cells and binds to B7-H2 (Yoshinaga, et al. (1999) Nature 402:827-832). ICOS is a positive coregulator, which is important for T cell activation, differentiation, and function (Yoshinaga, et al. (1999) supra; Dong, et al. (2001) Nature 409:97-101). In contrast, PD-1 (programmed death 1) negatively regulates T cell responses. PD-1 knockout mice develop lupus-like autoimmune disease or autoimmune dilated cardiomyopathy (Nishimura, et al. (1999) Immunity 11:141-151; Nishimura, et al. (2001) Science 291:319-322). The autoimmunity most likely results from the loss of signaling by both ligands PD-L1 and PD-L2. Recently, CD80 was identified as a second receptor for PD-L1 that transduces inhibitory signals into T cells (Butte, et al. (2007) Immunity 27:111-122).

The two inhibitory B7 family ligands, PD-L1 and PD-L2, have distinct expression patterns. PD-L2 is inducibly expressed on DCs and macrophages, whereas PD-L1 is broadly expressed on both hematopoietic cells and nonhematopoietic cell types (Okazaki & Honjo (2006) Immunology 27:195-201; Keir, et al. (2008) Annu. Rev. Immunol. 26:677-704). Consistent with the immune-suppressive role of PD-1 receptor, a study using PD-L1^{-/-} and PD-L2^{-/-} mice has shown that both ligands have overlapping roles in inhibiting T cell proliferation and cytokine production (Keir, et al. (2006) J. Exp. Med. 203:883-895). PD-L1 deficiency enhances disease progression in both the non-obese diabetic model of autoimmune diabetes and the mouse model of multiple sclerosis (experimental autoimmune encephalomyelitis (EAE); Anasari, et al. (2003) J. Exp. Med. 198:63-69; Salama, et al. (2003) J. Exp. Med. 198:71-78; Latchman, et al. (2004) Proc. Natl. Acad. Sci. USA. 101:10691-10696). PD-L1^{-/-} T cells produce elevated levels of the proinflammatory cytokines in both disease models. In addition, bone marrow chimera experiments have demonstrated that the tissue expression of PD-L1 (i.e., within pancreas) uniquely contributes to its capacity of regionally controlling inflammation (Keir, et al. (2006) supra; Keir, et al. (2007) J. Immunol. 179:5064-5070; Grabie, et al. (2007) Circulation. 116:2062-2071). PD-L1 is also highly expressed on placental syncytiotrophoblasts, which critically control the maternal immune responses to allogeneic fetus (Guleria, et al. (2005) J. Exp. Med. 202:231-237).

Consistent with its immune-suppressive role, PD-L1 potently suppresses antitumor immune responses and helps tumors evade immune surveillance. PD-L1 can induce apoptosis of infiltrating cytotoxic CD8⁺ T cells, which express a high level of PD-1 (Dong, et al. (2002) Nature 409:97-101; Dong & Chen (2003) J. Mol. Med. 81:281-287). Studies have shown that blocking the PD-L1-PD-1 signaling pathway, in conjunction with other immune therapies, prevents tumor progression by enhancing antitumor cytotoxic T lymphocyte activity and cytokine production (Iwai, et al. (2002) Proc. Natl. Acad. Sci. USA 99:12293-12297; Blank, et al. (2004) Cancer Res. 64:1140-1145; Blank, et al. (2005) Cancer Immunol. Immunother. 54:307-314; Geng, et al. (2006) Int. J. Cancer. 118:2657-2664). In addition, it has been shown that PD-L1 expression on dendritic cells promotes the induction of adaptive Foxp3⁺CD4⁺ regulatory T cells (aT_{reg} cells), and PD-L1 is a potent inducer of aT_{reg} cells within the tumor microenvironment (Wang, et al. (2008) Proc. Natl. Acad. Sci. USA. 105:9331-9336).

An additional immune regulatory ligand, referred to as V-domain Ig suppressor of T cell activation (VISTA) or PD-L3, has been recently identified as an upregulated molecule in a T cell transcriptional profiling screen. (Wang, et al. (2011) J. Exp. Med. 208:577; WO 2011/120013). It has been shown that the extracellular Ig domain of VISTA shares significant sequence homology with the B7 family ligands PD-L1 and PD-L2, albeit with unique structural features that distinguish it from the B7 family members.

VISTA is primarily expressed on hematopoietic cells, and VISTA expression is highly regulated on myeloid antigen-presenting cells (APCs) and T cells. Expression of VISTA on antigen presenting cells (APCs) suppresses T cell responses by engaging its counter-receptor on T cells during cognate interactions between T cells and APCs. VISTA blockade enhances T cell-mediated immunity in an autoimmune disease model, suggesting its unique and non-redundant role in controlling autoimmunity when compared with other inhibitory B7 family ligands such as PD-L1 and PD-L2. In addition, VISTA blockade enhances anti-tumor immunity and suppressed tumor growth in preclinical murine tumor models (WO 2011/120013). In this regard, therapeutic intervention of the VISTA inhibitory pathway represents a novel approach to modulate T cell-mediated immunity for treating diseases such as viral infection and cancer.

### SUMMARY OF THE INVENTION

The invention relates to synergistic therapies comprising the administration of a PD-1, PD-L1 or PD-L2 antagonist and a VISIA antagonist, e.g., an antagonistic anti-VISTA antibody or VISTA polypeptide fragment or conjugate containing and a antagonistic anti-PD-1 or anti-PD-L1 antibody or fragment or antagonistic PD-1 or PD-L1 or PD-L2 polypeptide or fragment or conjugate thereof, wherein these moieties respectively antagonize or inhibit the immunosuppressive effects of VISTA, PD-1, PD-L1 or PD-L2.

The invention further relates to synergistic therapies comprising the administration of a PD-1, PD-L1 or PD-L2 agonist and a VISIA agonist, e.g., an agonistic anti-VISTA antibody or VISTA polypeptide, e.g., VISTA-Ig fragment or conjugate containing and an agonistic anti-PD-1 or anti-PD-L1 antibody or fragment or agonistic PD-1 or PD-L1 or PD-L2 polypeptide or fragment or conjugate thereof, wherein these moieties respectively agonize or inhibit the immunosuppressive effects of VISTA, PD-1, PD-L1 or PD-L2.

In a further embodiment, the VISTA or PD-1 or PD-L1 or PD-L2 antagonist or agonist comprises another moiety that targets said peptide to a target site. The targeting moiety may be selected from an antibody or ligand that binds to an antigen, a receptor expressed by the target cell or an infectious agent.

In yet a further embodiment, the VISTA or PD-1 or PD-L1 or PD-L2 antagonist or agonist is attached to another moiety or another copy of said antagonist via a linker. The linker may be a peptide that permits the antagonist to interact with VISTA expressed on the surface of a target cell.

In a further embodiment, the VISTA or PD-1 or PD-L1 or PD-L2 antagonist or agonist is directly or indirectly attached to a detectable label or therapeutic agent.

In several of the embodiments, the VISTA or PD-1 or PD-L1 or PD-L2 antagonist binds to the extracellular domain of VISTA or PD-1 or PD-L1 or PD-L2 and disrupts its interaction with a VISTA receptor and/or reduces or inhibits VISTA or PD-1 or PD-L1 or PD-L2-mediated T cell suppression.

In several of the embodiments, the VISTA or PD-1 or PD-L1 or PD-L2 agonist binds to the extracellular domain of VISTA or PD-1 or PD-L1 or PD-L2 and promotes its interaction with a VISTA receptor and/or promotes VISTA or PD-1 or PD-L1 or PD-L2-mediated T cell suppression.

In one embodiment, the isolated VISTA or PD-1 or PD-L1 or PD-L2 antagonist combination elicits a synergistic or additive effect on anti-tumor and/or anti-viral activity.

In one embodiment, the isolated VISTA or PD-1 or PD-L1 or PD-L2 agonist combination elicits a synergistic or additive effect on autoimmune, allergic or inflammatory activity.

Additionally, the invention contemplates a synergistic suitable for therapeutic, prophylactic or diagnostic use comprising a therapeutically, prophylactically or diagnostically effective amount of the VISTA antagonist PD-1 or PD-L1 or PD-L2 antagonist.

Additionally, the invention contemplates a synergistic suitable for therapeutic, prophylactic or diagnostic use comprising a therapeutically, prophylactically or diagnostically effective amount of the VISTA agonist and PD-1 or PD-L1 or PD-L2 agonist.

In one embodiment, the composition further comprises a pharmaceutically acceptable carrier, diluent, solubilizer, preservative or mixture thereof.

In another embodiment, the composition further comprises another therapeutic agent, e.g., an anti-cancer agent, an anti-viral agent, a cytokine or an immune agonist. In a particular embodiment, the other therapeutic agent is selected from CTLA-4-Ig, anti-PD-1, PD-L1 or PD-L2 fusion proteins, and EGFR antagonists.

In another embodiment, the composition further comprises another therapeutic agent, e.g., an anti-autoimmune, allergic or anti-inflammatory agent, and an immune antagonist or suppressor. In a particular embodiment, the other therapeutic agent is selected from CTLA-4-Ig, anti-PD-1, PD-L1 or PD-L2 fusion proteins, and EGFR agonists.

In one embodiment, the composition is suitable for subcutaneous administration or intravenous administration.

In one embodiment, the invention provides a method for blocking, inhibiting or neutralizing T cell suppression, comprising administering to a subject in need thereof an effective amount of a combination of a VISTA antagonist VISTA and one of a PD-1 or PD-L1 or PD-L2 antagonist or a composition containing said antagonists.

In one embodiment, the invention provides a method for promoting T cell suppression, comprising administering to a subject in need thereof an effective amount of a combination of a VISTA agonist VISTA and one of a PD-1 or PD-L1 or PD-L2 agonist or a composition containing said agonists.

In another embodiment, the invention provides a method for stimulating an immune response in a subject, comprising administering to the subject in need thereof an effective amount of an isolated VISTA antagonist disclosed herein or a composition containing a VISTA antagonist and a PD-L1 or PD-1 or PD-L2 antagonist. Such a method may be used for treating cancer in a subject.

In another embodiment, the invention provides a method for inhibiting an immune response in a subject, comprising administering to the subject in need thereof an effective amount of an isolated VISTA tagonist disclosed herein or a composition containing a VISTA agonist and a PD-L1 or PD-1 or PD-L2 agonist. Such a method may be used for treating autoimmunity, allergy or inflammation in a subject.

The subject may have cancer and/or an infection selected from the group consisting of bacterial, viral, parasitic and fungal infections.

The bacterial infection may be caused by at least one bacterium selected from the group consisting of Bordetella, Borrelia, Brucella, Burkholderia, Campylobacter, Chlamydia, Clostridium, Corynebacterium, Enterobacter, Enterococcus, Erwinia, Escherichia, Francisella, Haemophilus, Heliobacter, Legionella, Leptospira, Listeria, Mycobacterium, Mycoplasma, Neisseria, Pasteurella, Pelobacter, Pseudomonas, Rickettsia, Salmonella, Serratia, Shigella, Staphylococcus, Streptococcus, Treponema, Vibrio, Yersinia and Xanthomonas.

The viral infection may be caused by at least one virus selected from the group consisting of Adenoviridae, Papillomaviridae, Polyomaviridae, Herpesviridae, Poxviridae, Hepadnaviridae, Parvoviridae, Astroviridae, Caliciviridae, Picornaviridae, Coronoviridae, Flaviviridae, Retroviridae, Togaviridae, Arenaviridae, Bunyaviridae, Filoviridae, Orthomyxoviridae, Paramyxoviridae, Rhabdoviridae, and Reoviridae. More specifically, the virus may be adenovirus, herpes simplex type I, herpes simplex type 2, Varicella-zoster virus, Epstein-barr virus, cytomegalovirus, herpesvirus type 8, papillomavirus, BK virus, JC virus, smallpox, Hepatitis B, bocavirus, parvovirus B19, astrovirus, Norwalk virus, coxsackievirus, Hepatitis A, poliovirus, rhinovirus, severe acute respiratory syndrome virus, Hepatitis C, yellow fever, dengue virus, West Nile virus, rubella, Hepatitis E, human immunodeficiency virus (HIV), influenza, guanarito virus, Junin virus, Lassa virus, Machupo virus, Sabia virus, Crimean-Congo hemorrhagic fever virus, ebola virus, Marburg virus, measles virus, mumps virus, parainfluenza, respiratory syncytial virus, human metapneumovirus, Hendra virus, Nipah virus, rabies, Hepatitis D, rotavirus, orbivirus, coltivirus or Banna virus.

The fungal infection may be selected from the group consisting of thrush, candidiasis, cryptococcosis, histoplasmosis, blastomycosis, aspergillosis, coccidioidomycosis, paracoccidiomycosis, sporotrichosis, zygomycosis, chromoblastomycosis, lobomycosis, mycetoma, onychomycosis, piedra pityriasis versicolor, tinea barbae, tinea capitis, tinea corporis, tinea cruris, tinea favosa, tinea nigra, tinea pedis, otomycosis, phaeohyphomycosis, or rhinosporidiosis.

The parasitic infection may be caused by at least one parasite selected from the group consisting of Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Toxoplasma gondii, Plasmodium vivax, Plasmodium ovale, Plasmodium malariae, Plasmodium falciparum, Trichomonas vaginalis, Histomonas meleagridis; Secementea; Trichuris trichiura, Ascaris lumbricoides, Enterobius vermicularis, Ancylostoma duodenale, Necator americanus, Strongyloides stercoralis, Wuchereria bancrofti, Dracunculus medinensis; blood flukes, liver flukes, intestinal flukes, lung flukes; Schistosoma mansoni, Schistosoma haematobium, Schistosoma japonicum, Fasciola hepatica, Fasciola gigantica, Heterophyes heterophyes, and Paragonimus westermani.

In another embodiment, the invention provides a method for enhancing anti-cancer or anti-tumor immunity, comprising administering to a subject in need thereof an effective amount of an isolated VISTA antagonist disclosed herein or a composition containing said isolated VISTA antagonist.

In another embodiment, the invention provides a method for treating or preventing cancer, inhibiting tumor invasion and/or cancer metastasis, comprising administering to a subject in need thereof an effective amount of an isolated VISTA antagonist disclosed herein or a composition containing said isolated VISTA antagonist.

The cancer may be selected from the group consisting of carcinoma, lymphoma, blastoma, sarcoma, leukemia, lymphoid malignancies, melanoma, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma, head and neck cancer, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; post-transplant lymphoproliferative disorder (PTLD), abnormal vascular proliferation associated with phakomatoses, edema (such as that associated with brain tumors), and Meigs' syndrome.

In yet another embodiment, the invention provides a method for treating or preventing a viral infection, comprising administering to a subject in need thereof an effective amount of an isolated VISTA antagonist disclosed herein or a composition containing said isolated VISTA antagonist.

These methods may further comprise the administration of another therapeutic agent, wherein said peptide and therapeutic may be separately or jointly administered, at the same or different times.

In one embodiment, the other therapeutic agent is an anti-cancer agent, an anti-viral or other anti-infectious agent, a cytokine or an immune agonist. Preferably, the other therapeutic agent is selected from CTLA-4-Ig, anti-PD-1, PD-L1 or PD-L2 fusion proteins, and EGFR antagonists.

Finally, the present invention also contemplates a method for mapping the active site of VISTA, comprising: (a) incubating an isolated VISTA fusion protein with an isolated VISTA antagonist comprising a peptide that is identical to the amino acid sequence of SEQ ID NO:1 (Ser-Ser-Ala-Cys-Asp-Trp-Ile-Lys-Arg-Ser-Cys-His), or which comprises a peptide having an amino acid sequence which differs from SEQ ID NO:1 by at most 2 amino acid residues or an multimer, conjugate, analog, derivative or mimetic thereof; and (b) determining the binding site of the isolated VISTA antagonist.

In one embodiment, the active site of VISTA binds to a VISTA receptor and mediates immune suppression.

In another embodiment, step (b) comprises domain deletion, domain swapping, amino acid mutagenesis, foot printing, NMR, X-ray crystallography or homology modeling.

### BRIEF DESCRIPTION OF THE DRAWINGS

FIG. 1 shows that a VISTA antagonist peptide (SEQ ID NO:1) significantly enhances the proliferation of T cells as compared to an anti-VISTA antibody (aVISTA) and an anti-PD-L1 antibody (aPDL1). Myeloid CD11B+ APCs were incubated with OT2 CD4+ T cells, antigen, and a monoclonal antibody (aVISTA or aPDL1) or AP1049. Proliferation of T cells was measured by tritium incorporation at 72 hours.

FIGS. 2A-B shows histological analysis of aged VISTA KO, PD-1 KO, and VISTA/PD-1 double KO mice. Necropsy was performed on 12 months old WT (n=16), VISTA KO (n=15), PD-1 KO (n=28), and VISTA/PD-1 double KO (n=25) mice. Organs were fixed, paraffin embedded, sectioned, and stained with H&E. Two representative H&E sections from lung, liver, and pancreas of the VISTA/PD-1 double KO mice were shown in (A). Clusters of tissue-infiltrating leukocytes were marked with black arrows. (Top row) Areas of necrotic tissues were marked with white arrows (Bottom row). All images are of 200× magnification. Scale bar: 50 microns. The inflammatory state of the tissues was evaluated based on a semi-quantitative method that scores the level of the leukocyte infiltration and tissue necrosis (B).

FIGS. 3A-F shows spontaneous T cell activation in the VISTA KO, PD-1 KO, and VISTA/PD-1 double KO mice. Splenic T cells were collected from age and gender-matched 6-7 months old WT (n=6), VISTA KO (n=4), PD-1 KO (n=6), and VISTA/PD-1 double KO (n=8) mice. The percentages of CD8+ and CD4+ T cells with activated phenotype (CD44hi CD62Llo) were quantified by flow cytometry. T cells were stimulated ex vivo overnight with soluble anti-CD3/CD28 mAbs, and their cytokine production (i.e. IFNγ, TNFα and IL-17A) was examined by intracellular staining. CD8+ T cell phenotypes were shown in A and B. CD4+ T cell phenotypes were shown in C-F. Representative results of at least three independent experiments were shown.

FIGS. 4A-D shows combined genetic deficiency of VISTA and PD-1 exacerbated autoimmune disease on the susceptible background. The CNS disease incidence (A) and mortality (B) were monitored in 2D2 TCR transgenic mice that were bred onto the VISTA KO, PD-1 KO, and the double KO genetic background. Representative H&E stained spinal cord section from paralyzed double KO mice was shown (C). Enlarged images show areas of extensive lymphocyte infiltration. Luxol fast blue staining of spinal cord sections confirmed extensive demyelination (D). 2D2-WT (n=30), 2D2-VISTA KO (n=42), 2D2-PD-1 KO (n=40), 2D2-VISTA/PD-1 double KO (n=37). Only one 2D2-WT mouse developed disease.

FIGS. 5A-D shows VISTA and the PD-1 collaboratively controlled antigen-specific T cell responses. 6-7 weeks old WT (n=8), VISTA KO (n=9), PD-1 KO (n=7), and VISTA/PD-1 double KO (n=6) mice were immunized with 50 μg soluble peptides OVA257-264 (A) or 2W1S (B) together with TLR3 agonist poly (I:C) (100 μg) as adjuvant. Splenocytes were harvested on Day +7 post immunization and re-stimulated with the respective peptides. IFNγ-producing cells were enumerated by the ELISPot assay. To stimulate T cells in vitro, CD11b+ CD11c+ DCs were sorted from WT, VISTA KO, PD-L1 KO, and VISTA/PD-L1 double KO mice, and incubated with naïve CD4+ OTII TCR transgenic T cells in the presence of cognate peptides OVA323-339 (10 ng/mL). [3H]-Thymidine was added to the culture for the last 8 hrs of the 72 hrs culture period for measuring T cell proliferation (C). The production of IFNγ was quantified from the culture supernatants by ELISA (D).

FIGS. 6A-D shows the engagement of both VISTA and PD-L1 during TCR activation maximally suppressed TCR signaling. To determine whether VISTA engagement impairs the recruitment of signaling adaptor protein LAT, DO11.10 hybridoma cells (100×106) were stimulated with plate-bound anti-CD3 mAb (2C11, 3 fÊg/ml), together with co-immobilized control-Ig (8 fÊg/ml) or VISTA-Ig fusion protein (8 fÊg/ml) for 10 min at 37° C., and lysed in situ. After removing the unbound cell lysates, plate-bound protein was eluted off the plate, and examined by Western blotting (A). To examine the effect of VISTA on the phosphorylation of TCR signaling molecules, CD25-CD4+ T cells were purified from naïve splenocytes and stimulated with plate-bound 2C11 (3 fÊg/ml) together with control-Ig (8 fÊg/ml) or VISTA-Ig (8 fÊg/ml) for 5 min at 37° C. Total cell lysates were prepared and the phosphorylation status of LAT, SLP76, PLC-fÁ1, Akt, and Erk1/2 was examined (B). To determine whether co-engagement of both VISTA and PD-L1 maximally suppresses LAT activation, DO11.10 cells were stimulated with plate-bound 2C11 (2.5fÊg/ml), together with control-Ig (10 fÊg/ml), or VISTA-Ig (5 fÊg/ml), or PD-L1-Ig (5 fÊg/ml), or both Ig fusion proteins. Cells were lysed after 10 min stimulation, and plate-bound proteins were recovered and examined as described above (C). To determine the synergistic effects of engaging both VISTA and PD-L1, pre-activated splenic CD4+ T cells were stimulated with plate-bound 2C11 (2.5 fÊg/ml) together with control-Ig (9 fÊg/ml), VISTA-Ig (3 fÊg/ml), PD-L1-Ig (6 fÊg/ml), or both Ig fusion proteins for 10 min at 37° C. Total cell lysates were harvested for Western blotting analysis (D). Representative results from 2-3 independent experiments were shown.

### DETAILED DESCRIPTION OF THE INVENTION

In order that the invention herein described may be fully understood, the following detailed description is set forth. Various embodiments of the invention are described in detail and may be further illustrated by the provided examples.

Unless defined otherwise, all technical and scientific terms used herein have the same meaning as those commonly understood by one of ordinary skill in the art to which this invention belongs. Although methods and materials similar or equivalent to those described herein may be used in the invention or testing of the present invention, suitable methods and materials are described herein. The materials, methods and examples are illustrative only, and are not intended to be limiting.

### Definitions

As used in the description herein and throughout the claims that follow, the meaning of “a,” “an,” and “the” includes plural reference unless the context clearly dictates otherwise.

“Antagonist,” as used herein, refers to a compound (preferably a polypeptide or antibody or fragment thereof) that opposed the physiological effects of another compound. For example, at the receptor level, an antagonist is a compound that opposes the receptor-associated response normally induced by another agent that binds to and activates the biological activity the receptor. Likewise, at the ligand level, an antagonist is a compound that opposes the ligand-associated response normally induced when the ligand binds to its target receptor and/or accessory factors. In a specific embodiment, a VISTA antagonist is a compound, e.g., a peptide or analog, derivative or mimetic thereof, that binds to VISTA and opposes one or more of its biological activities, e.g., VISTA-mediated T cell suppression and/or VISTA-mediated suppression of anti-tumor immunity, thereby enhancing T cell-mediated immunity and/or anti-tumor immunity.

“Agonist,” as used herein, refers to a compound (preferably a polypeptide or antibody or fragment thereof) that promotes the physiological effects of another compound. For example, at the receptor level, an agonist is a compound that promotes the receptor-associated response normally induced by another agent that binds to and activates the biological activity the receptor. Likewise, at the ligand level, an agonist is a compound that promotes the ligand-associated response normally induced when the ligand binds to its target receptor and/or accessory factors. In a specific embodiment, a VISTA agonist is a compound, e.g., a peptide or analog, derivative or mimetic thereof, that binds to VISTA or VISTA receptor and promotes one or more of its biological activities, e.g., VISTA-mediated T cell suppression and/or VISTA-mediated suppression of anti-tumor immunity, thereby inhibiting T cell-mediated immunity and/or autoimmunity, allergy or inflammation.

VISTA antagonist is a polypeptide or antibody that antagonizes VISTA activity, e.g., an antibody or fragment of VISTA or a conjugate.

PD-1 antagonist is a polypeptide or antibody that antagonizes PD-1 activity, e.g., an antibody or fragment of PD-1 or a conjugate.

PD-L1 antagonist is a polypeptide or antibody that antagonizes PD-L1 activity, e.g., an antibody or fragment of PD-L1 or a conjugate.

PD-L2 antagonist is a polypeptide or antibody that antagonizes PD-L2 activity, e.g., an antibody or fragment of PD-L1 or a conjugate.

VISTA agonist is a polypeptide or antibody that agonizes VISTA activity, e.g., an antibody or fragment of VISTA or a conjugate, e.g., VISTA-Ig fusion polypeptide.

PD-1 agonist is a polypeptide or antibody that agonizes PD-1 activity, e.g., an antibody or fragment of PD-1 or a conjugate, e.g., PD-1-Ig fusion polypeptide.

PD-L1 agonist is a polypeptide or antibody that agonizes PD-L1 activity, e.g., an antibody or fragment of PD-1 or a conjugate, e.g., PD-L1-Ig fusion polypeptide.

PD-L2 agonist is a polypeptide or antibody that antagonizes PD-L2 activity, e.g., an antibody or fragment of PD-L2 or a conjugate, e.g., PD-L2-Ig fusion polypeptide.

“Antigen presenting cell,” as used herein, refers broadly to professional antigen presenting cells (e.g., B lymphocytes, monocytes, dendritic cells, and Langerhans cells) as well as other antigen presenting cells (e.g., keratinocytes, endothelial cells, astrocytes, fibroblasts, and oligodendrocytes).

“Amino acid,” as used herein refers broadly to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified (e.g., hydroxyproline, γ-carboxyglutamate, and 0-phosphoserine.) Amino acid analogs refers to compounds that have the same basic chemical structure as a naturally occurring amino acid (i.e., a carbon that is bound to a hydrogen, a carboxyl group, an amino group), and an R group (e.g., homoserine, norleucine, methionine sulfoxide, methionine methyl sulfonium.) Analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Allergic disease,” as used herein, refers broadly to a disease involving allergic reactions. More specifically, an “allergic disease” is defined as a disease for which an allergen is identified, where there is a strong correlation between exposure to that allergen and the onset of pathological change, and where that pathological change has been proven to have an immunological mechanism. Herein, an immunological mechanism means that leukocytes show an immune response to allergen stimulation.

“Autoimmune disease” as used herein, refers broadly to a disease or disorder arising from and directed against an individual's own tissues or a co-segregate or manifestation thereof or resulting condition therefrom.

“Inflammatory disease” includes any disease associated with inflammation, e.g., chronic inflammation such as arthritis, rheumatoid arthritis, psoriatic arthritis, psoriasis, multiple sclerosis, etc.

“Cancer,” as used herein, refers broadly to any neoplastic disease (whether invasive or metastatic) characterized by abnormal and uncontrolled cell division causing malignant growth or tumor (e.g., unregulated cell growth.)

“Conservatively modified variants,” as used herein, applies to both amino acid and nucleic acid sequences, and with respect to particular nucleic acid sequences, refers broadly to conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. “Silent variations” are one species of conservatively modified nucleic acid variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) may be modified to yield a functionally identical molecule.

“Costimulatory receptor,” as used herein, refers broadly to receptors which transmit a costimulatory signal to an immune cell, e.g., CD28 or ICOS.

“Cytoplasmic domain,” as used herein, refers broadly to the portion of a protein which extends into the cytoplasm of a cell.

“Derivative” or “peptide derivative,” as used herein, contain a modification of one or more amino acid residues or a linker group or other covalently linked group. Non-limiting examples of derivatives include N-acyl derivatives of the amino terminal or of another free amino group, esters of the carboxyl terminal or of another free carboxyl or hydroxy group, amides of the carboxyl terminal or of another free carboxyl group produced by reaction with ammonia or with a suitable amine, glycosylated derivatives, hydroxylated derivatives, nucleotidylated derivatives, ADP-ribosylated derivatives, pegylated derivatives, phosphorylated derivatives, derivatives conjugated to lipophilic moieties, and derivatives conjugated to an antibody or other biological ligand. Also included among the chemical derivatives are those obtained by modification of the peptide bond —CO—NH—, for example by reduction to —CH₂—NH— or alkylation to —CO—N(alkyl)-. Preferred derivatisation include, but are not limited tom C-terminal amidation and N-terminal acetylation, which removes the negative charge of the C terminus or removes the positive charge at the N-terminus, respectively. Blocking of the C- or N-terminus, such as by C-terminal amidation or N-terminal acetylation, may improve proteolytic stability due to reduced susceptibility to exoproteolytic digestion. Peptide derivatives having a C-terminal amide are represented with “NH₂” at the C-terminus.

“Diagnostic,” as used herein, refers broadly to identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

“Diagnosing,” as used herein refers broadly to classifying a disease or a symptom, determining a severity of the disease, monitoring disease progression, forecasting an outcome of a disease and/or prospects of recovery. The term “detecting” may also optionally encompass any of the foregoing. Diagnosis of a disease according to the present invention may, in some embodiments, be affected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject.

“Effective amount,” as used herein, refers broadly to the amount of a compound, antibody, antigen, or cells that, when administered to a patient for treating a disease, is sufficient to effect such treatment for the disease. The effective amount may be an amount effective for prophylaxis, and/or an amount effective for prevention. The effective amount may be an amount effective to reduce, an amount effective to prevent the incidence of signs/symptoms, to reduce the severity of the incidence of signs/symptoms, to eliminate the incidence of signs/symptoms, to slow the development of the incidence of signs/symptoms, to prevent the development of the incidence of signs/symptoms, and/or effect prophylaxis of the incidence of signs/symptoms. The “effective amount” may vary depending on the disease and its severity and the age, weight, medical history, susceptibility, and pre-existing conditions, of the patient to be treated. The term “effective amount” is synonymous with “therapeutically effective amount” for purposes of this invention.

“Synergistic Effective amount,” as used herein, refers broadly to the amount of a compound, e.g., antibody, antigen, or cells that, when administered with another compound, e.g., antibody, antigen, or cells to a patient for treating a disease, is sufficient to elicit a greater than additive effect of the two compounds, e.g., a synergistic effect on promoting or inhibiting T cell immunity. The synergy may be 1-fold, 2, 3, 4 or 5-fold, 1-5 fold, 1-10 fold, 10-100-fold, or 1000 fold or more greater than the expected additive effect of both. The doses or amounts of both compounds may be varied to promote synergy, by use of different weight ratios of both, e.g., from 1:1, 1:2, 1:3, 1:4, 1:5, 1:10, 1:100, 1:1000 or more wherein the amount of either agonist or antagonist may be greater, equal or less to the other e.g., according to said weight ratios.

“Additive Effective amount,” as used herein, refers broadly to the amount of a compound, e.g., antibody, antigen, or cells that, when administered with another compound, e.g., antibody, antigen, or cells to a patient for treating a disease, is sufficient to elicit a greater effect than either administered alone, e.g., an additive effect on promoting or inhibiting T cell immunity, wherein such additive effect may be less than the expected combined effects of both compounds.

“Extracellular domain,” as used herein refers broadly to the portion of a protein that extend from the surface of a cell.

“Expression vector,” as used herein, refers broadly to any recombinant expression system for the purpose of expressing a nucleic acid sequence of the invention in vitro or in vivo, constitutively or inducibly, in any cell, including prokaryotic, yeast, fungal, plant, insect or mammalian cell. The term includes linear or circular expression systems. The term includes expression systems that remain episomal or integrate into the host cell genome. The expression systems can have the ability to self-replicate or not, i.e., drive only transient expression in a cell. The term includes recombinant expression cassettes which contain only the minimum elements needed for transcription of the recombinant nucleic acid.

“Homology,” as used herein, refers broadly to a degree of similarity between a nucleic acid sequence and a reference nucleic acid sequence or between a polypeptide sequence and a reference polypeptide sequence. Homology may be partial or complete. Complete homology indicates that the nucleic acid or amino acid sequences are identical. A partially homologous nucleic acid or amino acid sequence is one that is not identical to the reference nucleic acid or amino acid sequence. The degree of homology can be determined by sequence comparison. The term “sequence identity” may be used interchangeably with “homology.”

“Host cell,” as used herein, refers broadly to refer to a cell into which a nucleic acid molecule of the invention, such as a recombinant expression vector of the invention, has been introduced. Host cells may be prokaryotic cells (e.g., E. coli), or eukaryotic cells such as yeast, insect (e.g., SF9), amphibian, or mammalian cells such as CHO, HeLa, HEK-293, e.g., cultured cells, explants, and cells in vivo. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, progeny may not, in fact, be identical to the parent cell, but are still included within the scope of the term as used herein.

“Immune response,” as used herein, refers broadly to T cell-mediated and/or B cell-mediated immune responses that are influenced by modulation of T cell costimulation. Exemplary immune responses include B cell responses (e.g., antibody production) T cell responses (e.g., cytokine production, and cellular cytotoxicity) and activation of cytokine responsive cells, e.g., macrophages. As used herein, the term “down modulation” with reference to the immune response includes a diminution in any one or more immune responses, while the term “up modulation” with reference to the immune response includes an increase in any one or more immune responses. It will be understood that up modulation of one type of immune response may lead to a corresponding downmodulation in another type of immune response. For example, up modulation of the production of certain cytokines (e.g., IL-10) can lead to downmodulation of cellular immune responses.

“Inflammatory disease,” as used herein, refers broadly to chronic or acute inflammatory diseases.

“Detectable label” as used herein, refers broadly to a composition detectable by spectroscopic, photochemical, biochemical, immunochemical, chemical, or other physical means.

“Mimetic” or “peptidomimetic,” as used herein, refers to a fully or partially synthetic peptide that has the activity of a given peptide. Such a mimetic or peptidomimetic comprises one or more amino acid residues that is an artificial chemical mimetic of a corresponding naturally occurring amino acid, naturally occurring amino acid polymers and non-naturally occurring amino acid polymers.

Modifications of the VISTA and VISTA conjugate polypeptides described herein include, but are not limited to N-terminus modification, C-terminus modification, peptide bond modification (e.g., CH₂—NH, CH₂—S, CH₂—S═O, O═C—NH, CH₂—O, CH₂—CH₂, S═C—NH, CH═CH or CF═CH), backbone modifications, and residue modification, e.g., by the addition of carbohydrate residues to form glycoproteins, by the addition of chemical residues such as PEG and/or XTEN, etc. Methods for preparing peptidomimetic compounds are well known in the art. Martin, (2010).

“Nucleic acid” or “nucleic acid sequence,” as used herein, refers broadly to a deoxy-ribonucleotide or ribonucleotide oligonucleotide in either single- or double-stranded form. The term encompasses nucleic acids, i.e., oligonucleotides, containing known analogs of natural nucleotides. The term also encompasses nucleic-acid-like structures with synthetic backbones. Unless otherwise indicated, a particular nucleic acid sequence also implicitly encompasses conservatively modified variants thereof (e.g., degenerate codon substitutions) and complementary sequences, as well as the sequence explicitly indicated. The term nucleic acid is used interchangeably with gene, cDNA, mRNA, oligonucleotide, and polynucleotide.

“Polypeptide,” “peptide” and “protein,” are used interchangeably and refer broadly to a polymer of amino acid residues. The terms apply to amino acid polymers in which one or more amino acid residue is an analog or mimetic of a corresponding naturally occurring amino acid, as well as to naturally occurring amino acid polymers. The terms apply to amino acid polymers in which one or more amino acid residue is an artificial chemical mimetic of a corresponding naturally occurring amino acid, as well as to naturally occurring amino acid polymers and non-naturally occurring amino acid polymer. Polypeptides can be modified, e.g., by the addition of carbohydrate residues to form glycoproteins. The terms “polypeptide,” “peptide” and “protein” include glycoproteins, as well as non-glycoproteins.

“Prophylactically effective amount,” as used herein, refers broadly to the amount of a compound that, when administered to a patient for prophylaxis of a disease or prevention of the reoccurrence of a disease, is sufficient to effect such prophylaxis for the disease or reoccurrence. The prophylactically effective amount may be an amount effective to prevent the incidence of signs and/or symptoms. The “prophylactically effective amount” may vary depending on the disease and its severity and the age, weight, medical history, predisposition to conditions, preexisting conditions, of the patient to be treated.

“Prophylaxis,” as used herein, refers broadly to a course of therapy where signs and/or symptoms are not present in the patient, are in remission, or were previously present in a patient. Prophylaxis includes preventing disease occurring subsequent to treatment of a disease in a patient. Further, prevention includes treating patients who may potentially develop the disease, especially patients who are susceptible to the disease (e.g., members of a patent population, those with risk factors, or at risk for developing the disease).

“Recombinant” as used herein, refers broadly with reference to a product, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

“Sequence identity,” as used herein, refers broadly to a degree of similarity between a nucleic acid sequence and a reference nucleic acid sequence or between a polypeptide sequence and a reference polypeptide sequence. Sequence identity (also synonymous with “homology”) may be partial or complete. Complete sequence identity indicates that the nucleic acid or amino acid sequences are identical, i.e., 100% sequence identity. A partially homologous nucleic acid or amino acid sequence is one that is not identical to the reference nucleic acid or amino acid sequence. The degree of homology can be determined by sequence comparison, e.g., 60% identity, 70% identity, 80% identity, 90% identity, 95% identity, 97% identity, 98% identity, or 99% identity.

“Signs” of disease, as used herein, refers broadly to any abnormality indicative of disease, discoverable on examination of the patient; an objective indication of disease, in contrast to a symptom, which is a subjective indication of disease.

“Subject,” as used herein, refers broadly to any animal that is in need of treatment either to alleviate a disease state or to prevent the occurrence or reoccurrence of a disease state. Also, “subject” as used herein, refers broadly to any animal that has risk factors, a history of disease, susceptibility, symptoms, and signs, was previously diagnosed, is at risk for, or is a member of a patient population for a disease. The subject may be a clinical patient such as a human or a veterinary patient such as a companion, domesticated, livestock, exotic, or zoo animal. The term “subject” may be used interchangeably with the term “patient.”

“Symptoms” of disease as used herein, refers broadly to any morbid phenomenon or departure from the normal in structure, function, or sensation, experienced by the patient and indicative of disease.

“T cell,” as used herein, refers broadly to CD4+ T cells and CD8+ T cells. The term T cell also includes both T helper 1 type T cells and T helper 2 type T cells.

“Therapy,” “therapeutic,” “treating,” or “treatment”, as used herein, refers broadly to treating a disease, arresting, or reducing the development of the disease or its clinical symptoms, and/or relieving the disease, causing regression of the disease or its clinical symptoms. Therapy encompasses prophylaxis, treatment, remedy, reduction, alleviation, and/or providing relief from a disease, signs, and/or symptoms of a disease. Therapy encompasses an alleviation of signs and/or symptoms in patients with ongoing disease signs and/or symptoms (e.g., inflammation, pain). Therapy also encompasses “prophylaxis”. The term “reduced”, for purpose of therapy, refers broadly to the clinical significant reduction in signs and/or symptoms. Therapy includes treating relapses or recurrent signs and/or symptoms (e.g., inflammation, pain). Therapy encompasses but is not limited to precluding the appearance of signs and/or symptoms anytime as well as reducing existing signs and/or symptoms and eliminating existing signs and/or symptoms. Therapy includes treating chronic disease (“maintenance”) and acute disease. For example, treatment includes treating or preventing relapses or the recurrence of signs and/or symptoms (e.g., inflammation, pain).

“Transmembrane domain,” as used herein, refers broadly to an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In an embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta, et al. (1996) Annu. Rev. Neurosci. 19:235-263.

“Tumor,” as used herein, refers broadly to at least one cell or cell mass in the form of a tissue neoformation, in particular in the form of a spontaneous, autonomous and irreversible excess growth, which is more or less disinhibited, of endogenous tissue, which growth is as a rule associated with the more or less pronounced loss of specific cell and tissue functions. This cell or cell mass is not effectively inhibited, in regard to its growth, by itself or by the regulatory mechanisms of the host organism, e.g., melanoma or carcinoma. Tumor antigens not only include antigens present in or on the malignant cells themselves, but also include antigens present on the stromal supporting tissue of tumors including endothelial cells and other blood vessel components.

“Vector,” as used herein, refers broadly to a nucleic acid molecule capable of transporting another nucleic acid molecule to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. The techniques and procedures are generally performed according to conventional methods well known in the art and as described in various general and more specific references that are cited and discussed throughout the present specification. See, e.g., Sambrook, et al. (2001) Molec. Cloning: Lab. Manual [3^{rd} Ed] Cold Spring Harbor Laboratory Press. Standard techniques may be used for recombinant DNA, oligonucleotide synthesis, and tissue culture, and transformation (e.g., electroporation, lipofection). Enzymatic reactions and purification techniques may be performed according to manufacturer's specifications or as commonly accomplished in the art or as described herein.

The nomenclatures utilized in connection with, and the laboratory procedures and techniques of, analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, and delivery, and treatment of patients.

The VISTA, PD-1, PD-L1 or PD-L2 antagonists provided herein may be modified to add a therapeutic agent including, but not limited to, chemotherapeutic agents such as carboplatin, cisplatin, paclitaxel, gemcitabine, calicheamicin, doxorubicin, 5-fluorouracil, mitomycin C, actinomycin D, cyclophosphamide, vincristine, bleomycin, VEGF antagonists, EGFR antagonists, platins, taxols, irinotecan, 5-fluorouracil, gemcytabine, leucovorine, steroids, cyclophosphamide, melphalan, vinca alkaloids (e.g., vinblastine, vincristine, vindesine and vinorelbine), mustines, tyrosine kinase inhibitors, radiotherapy, sex hormone antagonists, selective androgen receptor modulators, selective estrogen receptor modulators, PDGF antagonists, TNF antagonists, IL-1 antagonists, interleukins (e.g., IL-12 or IL-2), IL-12R antagonists, Toxin conjugated monoclonal antibodies, tumor antigen specific monoclonal antibodies, Erbitux®, Avastin®, Pertuzumab, anti-CD20 antibodies, Rituxan®, ocrelizumab, ofatumumab, DXL625, Herceptin®, or any combination thereof. Toxic enzymes from plants and bacteria such as ricin, diphtheria toxin and Pseudomonas toxin may be conjugated to the VISTA antagonists to generate cell-type-specific-killing reagents. Youle, et al. (1980) Proc. Nat'l Acad. Sci. USA 77: 5483; Gilliland, et al. (1980) Proc. Nat'l Acad. Sci. USA 77: 4539; Krolick, et al. (1980) Proc. Nat'l Acad. Sci. USA 77: 5419.

Furthermore, the VISTA antagonists described herein may be conjugated to a radionuclide that emits alpha or beta particles (e.g., radioimmunoconjuagtes). Such radioactive isotopes include but are not limited to beta-emitters such as phosphorus-32 (³²P), scandium-47 (⁴⁷Sc), copper-67 (⁶⁷Cu), gallium-67 (⁶⁷Ga), yttrium-88 (⁸⁸Y), yttrium-90 (⁹⁰Y), iodine-125 (¹²⁵I), iodine-131 (¹³¹I), samarium-153 (¹⁵³Sm), lutetium-177 (¹⁷⁷Lu), rhenium-186 (¹⁸⁶Re), rhenium-188 (¹⁸⁸Re), and alpha-emitters such as astatine-211 (²¹¹At), lead-212 (²¹²Pb), bismuth-212 (²¹²Bi), bismuth-213 (²¹³Bi) or actinium-225 (²²⁵Ac).

Methods are known in the art for conjugating a VISTA antagonist described herein to a label, such as those methods described by Hunter, et al (1962) Nature 144: 945; David, et al. (1974) Biochemistry 13: 1014; Pain, et al. (1981) J. Immunol. Meth. 40: 219; and Nygren (1982) Histochem and Cytochem, 30: 407.

Additionally, the VISTA antagonists described herein may comprise another moiety, i.e., a “targeting moiety,” that targets the antagonist peptide to a target site (such as a cancer cell, a tumor, a virally-infected cell, etc). The targeting moiety may be selected from an antibody or ligand that binds to an antigen, a receptor expressed by the target cell or an infectious agent.

The VISTA antagonist (as well as multimers, conjugates, analogs, derivatives and mimetics thereof) may also be directly or indirectly attached to an immunoglobulin polypeptide or a fragment thereof, e.g., a antibody constant region.

A “conjugate,” as used herein, refers to a compound having at least one isolated VISTA antagonist peptide and one immunoglobulin polypeptide or a fragment thereof, e.g., antibody constant region, joined at the polypeptide level, with or without the use of a linker. A conjugate may be a fusion polypeptide produced as the result of joining at the nucleic acid level of genes encoding at least one natriuretic peptide and one antibody constant region, with or without a coding sequence for a peptide linker.

Such VISTA antagonist peptide-antibody conjugates may have a higher serum stability, e.g., at least 20%, preferably at least 30%, 50%, 80%, 100%, 200% or more, increase in the serum half-life when compared with the antagonist peptide without the antibody constant region under the same conditions. A human antibody, e.g., a human IgG, such as IgG1, IgG2, IgG3 or IgG4, is frequently used to derive a constant region or a fragment thereof for the purpose of making a natriuretic peptide conjugate of this invention.

As used herein, an “antibody (or immunoglobulin) constant region” refers to a polypeptide that corresponds to at least a portion of the constant region of an antibody heavy chain or light chain, such portion including at least one constant domain (e.g., the constant domain of CL or one of the constant domains of C_{H}). For example, an “antibody constant region” used for making the conjugates of this invention may be derived from an antibody heavy chain and include two out of three (C_{H}2 and C_{H}3 for IgA, IgD, and IgG) or three out of four (C_{H}2, CH3, and CH4, for IgE and IgM) constant domains; the first constant domain (C_{H}I) may be present in some cases but may be excluded in others. Such an antibody constant region can be obtained by a variety of means, e.g., by a recombinant method or synthetic method, or by purification subsequent to enzymatic digestion, for instance, pepsin or papain digestion of an intact antibody or an antibody heavy or light chain.

Further encompassed by this term as used in this application are polypeptides having a substantial sequence identity (for instance, at least 80%, 85%, 90%, 95% or more) to the corresponding amino acid sequence of an antibody heavy or light chain constant region or a portion thereof that contains at least one constant domain nearest to the C-terminus of the antibody chain, so long as the presence of such an “antibody constant region” in a VISTA antagonist peptide-antibody constant region conjugate renders the conjugate a higher serum stability.

Additionally, the peptide, multimer, conjugate, analog, derivative or mimetic may be modified to increase certain properties, e.g., biological half life. Various approaches are possible including, but not limited to, N-terminal modification/conjugation (e.g., lipidation or acetylation), C-terminal modification/conjugation (e.g., lipidation or acetylation), amino acid substitutions (i.e., substitution of natural amino acid with unnatural amino acids, such as D-conformation, N-methylation, tetra-substitution, beta-amino acids, etc.), peptide backbone modifications (e.g., chemical modification of peptide bonds, such as simple reductions or replacement of carbonyl or amide groups with esters, sulfides and alkyls), side chain modifications and/or cyclization (e.g., disulfide bond formation).

In one embodiment, the peptide may be pegylated to, e.g., increase the biological (e.g., serum) half life of the antibody. To pegylate a peptide, typically it is reacted with polyethylene glycol (PEG), such as a reactive ester or aldehyde derivative of PEG, under conditions in which one or more PEG groups become attached to the peptide. Preferably, the pegylation is carried out via an acylation reaction or an alkylation reaction with a reactive PEG molecule (or an analogous reactive water-soluble polymer.

Similarly, in another embodiment, a peptide, multimer, conjugate, analog, derivative or mimetic may be modified by conjugation of polysialic acid (PSA) to increase half-life.

Additionally, the peptide, multimer, conjugate, analog, derivative or mimetic may be modified, e.g., genetically fused or chemically conjugated, to comprise extended recombinant polypeptide (XTEN), through a process called XTENylation, to improve its half life. XTEN is a long, hydrophilic, and unstructured protein-based polymer of 864 amino acids. See, e.g., WO 2013/130683 which is herein expressly incorporated by reference in its entirety. When attached to a molecule of interest, greatly increases the effective size of the molecule, thereby prolonging its presence in serum by slowing kidney clearance in a manner analogous to that of PEG. In addition to slowing kidney clearance, attachment to XTEN can also inhibit receptor-mediated clearance by reducing the ligand's affinity for its receptor. XTEN coupling chemistries include, but are not limited to, Thiol-XTEN; Maleimide-XTEN; Alkyne-XTEN; and Iodoacetyl-XTEN.

Moreover, the peptide, multimer, conjugate, analog, derivative or mimetic may be modified with recombinant albumin, e.g., Novozymes Recombumin®, to improve half life. The peptide can be genetically fused or chemically conjugated to a recombinant albumin using standard protocols.

Furthermore, the peptide, multimer, conjugate, analog, derivative or mimetic may be modified by the addition and/or removal of specific amino acids to and/or from the peptide. For example, a number of specific amino acids may be added to the peptide, thereby strengthening or tightening its molecular structure to make it less susceptible to biological degradation and, thus, providing a longer life-span in the blood, using, e.g., Zealand Structure Induced Probe (SIP®) tail technology.

Yet another exemplary method for improving the stability and therapeutic potential of peptides, analogs, derivatives or mimetics is multimerization. For example, a multimer may comprise two or more copies, e.g., 2, 3, 4, 5, 6, 7 or more, of the isolated VISTA antagonist or variant thereof. Multimers include both homomultimers and heteromultimers. Multimerization can result in increased peptide stability, higher binding strength (due to multiple valencies in the molecule), and/or improved pharmacokinetic properties.

Another exemplary approach for improving the stability and, thus, therapeutic potential of the VISTA antagonist peptides, multimers, conjugates, analogs, derivatives or mimetics disclosed herein is the addition of acetyl groups to the N and/or C terminus of the peptide. Acetylation may protect the peptide from exopeptidases, thereby extending the half-life of the peptide.

### Production of VISTA Antagonists

The peptide multimer, conjugate, analog, derivative or mimetic can be produced and isolated using any method known in the art. Peptides can be synthesized, whole or in part, using chemical methods known in the art (see, e.g., Caruthers (1980) Nucleic Acids Res. Symp. Ser. 215-223; Horn (1980) Nucleic Acids Res. Symp. Ser. 225-232; and Banga (1995) Therapeutic Peptides and Proteins, Formulation, Processing and Delivery Systems, Technomic Publishing Co., Lancaster, Pa.). Peptide synthesis can be performed using various solid-phase techniques (see, e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the manufacturer's instructions.

Individual synthetic residues and peptides incorporating mimetics can be synthesized using a variety of procedures and methodologies known in the art (see, e.g., Organic Syntheses Collective Volumes, Gilman, et al. (Eds) John Wiley & Sons, Inc., NY). Peptides and peptide mimetics can also be synthesized using combinatorial methodologies. Techniques for generating peptide and peptidomimetic libraries are well-known, and include, for example, multipin, tea bag, and split-couple-mix techniques (see, for example, al-Obeidi (1998) Mol. Biotechnol. 9:205-223; Hruby (1997) Curr. Opin. Chem. Biol. 1:114-119; Ostergaard (1997) Mol. Divers. 3:17-27; and Ostresh (1996) Methods Enzymol. 267:220-234). Modified peptides can be further produced by chemical modification methods (see, for example, Belousov (1997) Nucleic Acids Res. 25:3440-3444; Frenkel (1995) Free Radic. Biol. Med. 19:373-380; and Blommers (1994) Biochemistry 33:7886-7896).

Alternatively, a peptide of this invention can be prepared in recombinant protein systems using polynucleotide sequences encoding the peptides. By way of illustration, a nucleic acid molecule encoding a peptide of the invention is introduced into a host cell, such as bacteria, yeast or mammalian cell, under conditions suitable for expression of the peptide, and the peptide is purified or isolated using methods known in the art. See, e.g., Deutscher et al. (1990) Guide to Protein Purification: Methods in Enzymology Vol. 182, Academic Press. In particular embodiments, the peptide, or analog, derivative or mimetic thereof is isolated and/or purified to homogeneity (e.g. greater than 90% purity).

It is contemplated that the peptide disclosed herein can be used as a lead compound for the design and synthesis of compounds with improved efficacy, clearance, half-lives, and the like.

One approach includes structure-activity relationship (SAR) analysis (e.g., NMR analysis) to determine specific binding interactions between the peptide and VISTA to facilitate the development of more efficacious agents. Agents identified in such SAR analysis or from agent libraries can then be screened for their ability to, e.g., decrease the activity of VISTA and/or enhance T cell proliferation.

### Pharmaceutical Compositions

The VISTA antagonist peptide, multimer, conjugate, analog, derivative and mimetic thereof described herein can be provided in a pharmaceutical composition.

A “pharmaceutical composition” refers to a chemical or biological composition suitable for administration to a mammal. Such compositions may be specifically formulated for administration via one or more of a number of routes, including but not limited to buccal, epicutaneous, epidural, inhalation, intraarterial, intracardial, intracerebroventricular, intradermal, intramuscular, intranasal, intraocular, intraperitoneal, intraspinal, intrathecal, intravenous, oral, parenteral, rectally via an enema or suppository, subcutaneous, subdermal, sublingual, transdermal, and transmucosal. In addition, administration may occur by means of injection, powder, liquid, gel, drops, or other means of administration.

A “pharmaceutical excipient” or a “pharmaceutically acceptable excipient” is a carrier, usually a liquid, in which an active therapeutic agent is formulated. In one embodiment of the invention, the active therapeutic agent is a humanized antibody described herein, or one or more fragments thereof. The excipient generally does not provide any pharmacological activity to the formulation, though it may provide chemical and/or biological stability, and release characteristics. Exemplary formulations may be found, for example, in Grennaro (2005) [Ed.] Remington: The Science and Practice of Pharmacy [21^{st} Ed.]

Pharmaceutical compositions typically must be sterile and stable under the conditions of manufacture and storage. The invention contemplates that the pharmaceutical composition is present in lyophilized form. The composition may be formulated as a solution, microemulsion, liposome, or other ordered structure suitable to high drug concentration. The carrier may be a solvent or dispersion medium containing, for example, water, ethanol, polyol (for example, glycerol, propylene glycol, and liquid polyethylene glycol), and suitable mixtures thereof. The invention further contemplates the inclusion of a stabilizer in the pharmaceutical composition.

The VISTA antagonist peptide, multimer, conjugate, analog, derivative and mimetic thereof described herein may be formulated into pharmaceutical compositions of various dosage forms. To prepare the pharmaceutical compositions of the invention, at least one VISTA antagonist as the active ingredient may be intimately mixed with appropriate carriers and additives according to techniques well known to those skilled in the art of pharmaceutical formulations. See Grennaro (2005) [Ed.] Remington: The Science and Practice of Pharmacy [21^{st} Ed.] For example, the antagonists described herein may be formulated in phosphate buffered saline pH 7.2 and supplied as a 5.0 mg/mL clear colorless liquid solution.

Similarly, compositions for liquid preparations include solutions, emulsions, dispersions, suspensions, syrups, and elixirs, with suitable carriers and additives including but not limited to water, alcohols, oils, glycols, preservatives, flavoring agents, coloring agents, and suspending agents. Typical preparations for parenteral administration comprise the active ingredient with a carrier such as sterile water or parenterally acceptable oil including but not limited to polyethylene glycol, polyvinyl pyrrolidone, lecithin, arachis oil or sesame oil, with other additives for aiding solubility or preservation may also be included. In the case of a solution, it may be lyophilized to a powder and then reconstituted immediately prior to use. For dispersions and suspensions, appropriate carriers and additives include aqueous gums, celluloses, silicates, or oils.

For each of the recited embodiments, the VISTA antagonist peptides, multimers, conjugates, analogs, derivatives and mimetics thereof may be administered by a variety of dosage forms. Any biologically-acceptable dosage form known to persons of ordinary skill in the art, and combinations thereof, are contemplated. Examples of such dosage forms include, without limitation, reconstitutable powders, elixirs, liquids, solutions, suspensions, emulsions, powders, granules, particles, microparticles, dispersible granules, cachets, inhalants, aerosol inhalants, patches, particle inhalants, implants, depot implants, injectables (including subcutaneous, intramuscular, intravenous, and intradermal), infusions, and combinations thereof.

In many cases, it will be preferable to include isotonic agents, e.g., sugars, polyalcohols such as mannitol, sorbitol, or sodium chloride in the composition. Prolonged absorption of the injectable compositions may be brought about by including in the composition an agent which delays absorption, e.g., monostearate salts and gelatin. Moreover, the compounds described herein may be formulated in a time release formulation, e.g. in a composition that includes a slow release polymer. The VISTA and VISTA conjugate may be prepared with carriers that will protect the compound against rapid release, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

A person of skill in the art would be able to determine an effective dosage and frequency of administration through routine experimentation, for example guided by the disclosure herein and the teachings in Goodman, et al. (2011) Goodman & Gilman's The Pharmacological Basis of Therapeutics [12^{th} Ed.]; Howland, et al. (2005) Lippincott's Illustrated Reviews: Pharmacology [2^{nd} Ed.]; and Golan, (2008) Principles of Pharmacology: The Pathophysiologic Basis of Drug Therapy [2^{nd} Ed.] See, also, Grennaro (2005) [Ed.] Remington: The Science and Practice of Pharmacy [21^{st} Ed.]

The compositions described herein may be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred routes of administration are intravenous injection or infusion. The administration can be local, where the composition is administered directly, close to, in the locality, near, at, about, or in the vicinity of, the site(s) of disease, e.g., tumor, or systemic, wherein the composition is given to the patient and passes through the body widely, thereby reaching the site(s) of disease. Local administration (e.g., injection) may be accomplished by administration to the cell, tissue, organ, and/or organ system, which encompasses and/or is affected by the disease, and/or where the disease signs and/or symptoms are active or are likely to occur (e.g., tumor site). Administration can be topical with a local effect, composition is applied directly where its action is desired (e.g., tumor site).

For each of the recited embodiments, the compounds can be administered by a variety of dosage forms as known in the art. Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds which can be included by admixture are, for example, medically inert ingredients (e.g., solid and liquid diluent), such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

In further embodiments, the present invention provides kits including one or more containers comprising pharmaceutical dosage units comprising an effective amount of one or more VISTA antagonists of the present invention. Kits may include instructions, directions, labels, marketing information, warnings, or information pamphlets.

The amount of VISTA antagonist in a therapeutic composition according to any embodiments of this invention may vary according to factors such as the disease state, age, gender, weight, patient history, risk factors, predisposition to disease, administration route, pre-existing treatment regime (e.g., possible interactions with other medications), and weight of the individual. Dosage regimens may be adjusted to provide the optimum therapeutic response. For example, a single bolus may be administered, several divided doses may be administered over time, or the dose may be proportionally reduced or increased as indicated by the exigencies of therapeutic situation.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of antibodies, and fragments thereof, calculated to produce the desired therapeutic effect in association with the required pharmaceutical carrier. The specification for the dosage unit forms of the invention are dictated by and directly dependent on the unique characteristics of the antibodies, and fragments thereof, and the particular therapeutic effect to be achieved, and the limitations inherent in the art of compounding such an antibodies, and fragments thereof, for the treatment of sensitivity in individuals. In therapeutic use for treatment of conditions in mammals (e.g., humans) for which the antibodies and fragments thereof of the present invention or an appropriate pharmaceutical composition thereof are effective, the antibodies and fragments thereof of the present invention may be administered in an effective amount. The dosages as suitable for this invention may be a composition, a pharmaceutical composition or any other compositions described herein.

The dosage may be administered as a single dose, a double dose, a triple dose, a quadruple dose, and/or a quintuple dose. The dosages may be administered singularly, simultaneously, and sequentially.

The dosage form may be any form of release known to persons of ordinary skill in the art. The compositions of the present invention may be formulated to provide immediate release of the active ingredient or sustained or controlled release of the active ingredient. In a sustained release or controlled release preparation, release of the active ingredient may occur at a rate such that blood levels are maintained within a therapeutic range but below toxic levels over an extended period of time (e.g., 4 to 24 hours). The preferred dosage forms include immediate release, extended release, pulse release, variable release, controlled release, timed release, sustained release, delayed release, long acting, and combinations thereof, and are known in the art.

As defined herein, a therapeutically effective amount of VISTA antagonist peptide, analog, derivative or mimetic thereof (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a peptide can include a single treatment or, preferably, can include a series of treatments.

In a preferred example, a subject is treated with peptide, analog, derivative or mimetic thereof in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

It will be appreciated that the pharmacological activity of the compositions may be monitored using standard pharmacological models that are known in the art. Furthermore, it will be appreciated that the compositions comprising a VISTA antagonist may be incorporated or encapsulated in a suitable polymer matrix or membrane for site-specific delivery, or may be functionalized with specific targeting agents capable of effecting site specific delivery. These techniques, as well as other drug delivery techniques are well known in the art. Determination of optimal dosages for a particular situation is within the capabilities of those skilled in the art. See, e.g., Grennaro (2005) [Ed.] Remington: The Science and Practice of Pharmacy [21^{st} Ed.]

A peptide or analog, derivative or mimetic of this invention can be co-formulated with and/or coadministered with one or more additional therapeutic agents (e.g., an anti-cancer agent, an anti-viral agent, a cytokine and/or an immune agonist). Such combination therapies may require lower dosages of the peptide or analog, derivative or mimetic and/or the co-administered agents, thus avoiding possible toxicities or complications associated with the various monotherapies. There are a number of agents that may be advantageously combined with peptide or analog, derivative or mimetic of the invention and the selection of such agents will depend on the intended disease or condition to be treated. For example, the present invention includes combination therapies composed of a peptide or multimer, conjugate, analog, derivative or mimetic of the invention that is capable of inducing or promoting a response against a cancerous or pre-cancerous condition and at least one anti-cancer agent. Accordingly, in particular embodiments, the instant peptide or analog, derivative or mimetic is used as an adjuvant therapy in the treatment of cancer. As another example, the invention embraces combination therapies that include a peptide or analog, derivative or mimetic of the invention that is capable of inducing or promoting a therapeutic response against a viral infection and at least one anti-viral agent. Exemplary therapeutic agents that may be contained in the compositions comprising the VISTA antagonist peptide, multimer, conjugate, analog, derivative or mimetic include, e.g., CTLA-4-Ig, anti-PD-1, PD-L1 or PD-L2 fusion proteins and EGFR antagonists.

Anti-cancer agents include, but are not limited to, cytotoxic agents such as Vinca alkaloid, taxanes, and topoisomerase inhibitors; antisense nucleic acids such as augmerosen/G3139, LY900003 (ISIS 3521), ISIS 2503, OGX-011 (ISIS 112989), LE-AON/LEraf-AON (liposome encapsulated c-raf antisense oligonucleotide/ISIS-5132), MG98, and other antisense nucleic acids that target PKCα, clusterin, IGFBPs, protein kinase A, cyclin D1, or Bcl-2; anticancer nucleozymes such as angiozyme (Ribozyme Pharmaceuticals); tumor suppressor-encoding nucleic acids such as a p53, BRCA1, RB1, BRCA2, DPC4 (Smad4), MSH2, MLH1, and DCC; oncolytic viruses such as oncolytic adenoviruses and herpes viruses; anti-cancer immunogens such as a cancer antigen/tumor-associated antigen, e.g., an epithelial cell adhesion molecule (Ep-CAM/TACSTDi), mucin 1 (MUCi), carcinoembryonic antigen (CEA), tumor-associated glycoprotein 72 (TAG-72), gp100, Melan-A, MART-1, KDR, RCAS1, MDA7, cancer-associated viral vaccines, tumor-derived heat shock proteins, and the like; anti-cancer cytokines, chemokines, or combination thereof; inhibitors of angiogenesis, neovascularization, and/or other vascularization; and/or any other conventional anticancer agent including fluoropyrimidiner carbamates, non-polyglutamatable thymidylate synthase inhibitors, nucleoside analogs, antifolates, topoisomerase inhibitors, polyamine analogs, mTOR inhibitors, alkylating agents, lectin inhibitors, vitamin D analogs, carbohydrate processing inhibitors, anti-metabolism folate antagonists, thumidylate synthase inhibitors, antimetabolites, ribonuclease reductase inhibitors, dioxolate nucleoside analogs, and chemically modified tetracyclines.

Anti-viral agents of use in the invention include, but are not limited to, protease inhibitors (e.g., acyclovir) in the context of HIV treatment or an anti-viral antibody (e.g., an anti-gp41 antibody in the context of HIV treatment; an anti-CD4 antibody in the context of the treatment of CMV, etc.). Numerous other types of anti-viral agents are known in the art.

Toxicity and therapeutic efficacy of the peptide or analog, derivative or mimetic can be determined by standard pharmaceutical procedures in cell cultures or experimental animals. The data obtained from the cell culture assays and animal studies can be used in formulating a range of dosage for use in humans. The dosage of such agents lies preferably within a range of circulating concentrations that include the ED₅₀ with little or no toxicity. The dosage may vary within this range depending upon the dosage form employed and the route of administration utilized. For any agent used in the methods of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Use of VISTA Antagonists and Compositions Comprising the Same

The peptide or analog, derivative or mimetic of this invention finds use in inhibiting the activity of VISTA (i.e., PD-L3) thereby upregulating immune responses. Upregulation of immune responses can be in the form of enhancing an existing immune response or eliciting an initial immune response. For example, enhancing an immune response through inhibition of VISTA activity is useful in the prevention and/or treatment of infections with microbes, e.g., bacteria, viruses, or parasites, or in cases of immunosuppression and cancer.

Accordingly, the present invention includes prophylactic and therapeutic methods for the prevention and treatment of cancer and infectious disease. Terms such as “treat,” “treating” and “treatment” herein refer to the delivery of an effective amount of a peptide or analog, derivative or mimetic of this invention with the purpose of easing, ameliorating, or eradicating (curing) such symptoms or disease states already developed. The terms “prevent,” “preventing” and “prevention” refer to the delivery of an effective amount of a peptide or analog, derivative or mimetic of this invention with the purpose of preventing any symptoms or disease state to develop. Thus, these terms are meant to include prophylactic treatment.

Accordingly to one embodiment, the invention provides a method of treating or preventing cancer, inhibiting tumor invasion and/or cancer metastasis by administering to a subject in need thereof, such as a mammalian subject, preferably a human subject, an effective amount of an isolated VISTA antagonist disclosed herein or a composition containing said isolated VISTA antagonist. Optionally the subject has one or more precancerous lesions or is predisposed to cancer, e.g., as a result of genetic mutation, family history or exposure to a carcinogenic agent. In another embodiment the invention provides a method of treating cancer in subject, such as a mammalian subject, preferably a human subject, such as a human subject, who optionally has a detectable level of cancer cells. In accordance with these embodiments, the subject is administered a peptide or analog, derivative or mimetic of this invention in an amount sufficient to detectably reduce the development or progression of the cancer in the subject.

Cancers are generally composed of single or several clones of cells that are capable of partially independent growth in a host (e.g., a benign tumor) or fully independent growth in a host (malignant cancer). Cancer cells are cells that divide and reproduce abnormally with uncontrolled growth.

Cancer cells arise from host cells via neoplastic transformation (i.e., carcinogenesis). Terms such as “preneoplastic,” “premalignant” and “precancerous” with respect to the description of cells and/or tissues herein refer to cells or tissues having a genetic and/or phenotypic profile that signifies a significant potential of becoming cancerous. Usually such cells can be characterized by one or more differences from their nearest counterparts that signal the onset of cancer progression or significant risk for the start of cancer progression. Such precancerous changes, if detectable, can usually be treated with excellent results.

In general, a precancerous state will be associated with the incidence of neoplasm(s) or preneoplastic lesion(s). Examples of known and likely preneoplastic tissues include ductal carcinoma in situ (DCIS) growths in breast cancer, cervical intra-epithelial neoplasia (CIN) in cervical cancer, adenomatous polyps of colon in colorectal cancers, atypical adenomatous hyperplasia in lung cancers, and actinic keratosis (AK) in skin cancers. Pre-neoplastic phenotypes and genotypes for various cancers, and methods for assessing the existence of a preneoplastic state in cells, have been characterized. See, e.g., Medina (2000) J. Mammary Gland Biol. Neoplasia 5(4):393-407; Krishnamurthy, et al. (2002) Adv. Anat. Pathol. 9(3):185-97; Ponten (2001) Eur. J. Cancer Suppl 8:S97-113; Niklinski, et al. (2001) Eur. J. Cancer Prev. 10(3):213-26; Walch, et al. Pathobiology (2000) 68(1):9-17; Busch (1998) Cancer Surv. 32:149-79.

Gene expression profiles can increasingly be used to differentiate between normal, precancerous, and cancer cells. For example, familial adenomatous polyposis genes prompt close surveillance for colon cancer; mutated p53 tumor-suppressor gene flags cells that are likely to develop into aggressive cancers; osteopontin expression levels are elevated in premalignant cells, and increased telomerase activity also can be a marker of a precancerous condition (e.g., in cancers of the bladder and lung). In one aspect, the invention relates to the treatment of precancerous cells. In another aspect, the invention relates to the preparation of medicaments for treatment of precancerous cells.

In general, a peptide or analog, derivative or mimetic of this invention can be used to treat subjects suffering from any stage of cancer (and to prepare medicaments for reduction, delay, or other treatment of cancer). Effective treatment of cancer (and thus the reduction thereof) can be detected by any variety of suitable methods. Methods for detecting cancers and effective cancer treatment include clinical examination (symptoms can include swelling, palpable lumps, enlarged lymph nodes, bleeding, visible skin lesions, and weight loss); imaging (X-ray techniques, mammography, colonoscopy, computed tomography (CT and/or CAT) scanning, magnetic resonance imaging (MRI), etc.); immunodiagnostic assays (e.g., detection of CEA, AFP, CA125, etc.); antibody-mediated radioimaging; and analyzing cellular/tissue immunohistochemistry. Other examples of suitable techniques for assessing a cancerous state and effective cancer treatment include PCR and RT-PCR (e.g., of cancer cell associated genes or “markers”), biopsy, electron microscopy, positron emission tomography (PET), computed tomography, magnetic resonance imaging (MRI), karyotyping and other chromosomal analysis, immunoassay/immunocytochemical detection techniques (e.g., differential antibody recognition), histological and/or histopathologic assays (e.g., of cell membrane changes), cell kinetic studies and cell cycle analysis, ultrasound or other sonographic detection techniques, radiological detection techniques, flow cytometry, endoscopic visualization techniques, and physical examination techniques.

In general, delivering a peptide or analog, derivative or mimetic of this invention to a subject (either by direct administration or expression from a nucleic acid) can be used to reduce, treat, prevent, or otherwise ameliorate any aspect of cancer in a subject. In this respect, treatment of cancer can include, e.g., any detectable decrease in the rate of normal cells transforming to neoplastic cells (or any aspect thereof), the rate of proliferation of pre-neoplastic or neoplastic cells, the number of cells exhibiting a pre-neoplastic and/or neoplastic phenotype, the physical area of a cell media (e.g., a cell culture, tissue, or organ) containing pre-neoplastic and/or neoplastic cells, the probability that normal cells and/or preneoplastic cells will transform to neoplastic cells, the probability that cancer cells will progress to the next aspect of cancer progression (e.g., a reduction in metastatic potential), or any combination thereof. Such changes can be detected using any of the above-described techniques or suitable counterparts thereof known in the art, which typically are applied at a suitable time prior to the administration of a therapeutic regimen so as to assess its effectiveness. Times and conditions for assaying whether a reduction in cancer has occurred will depend on several factors including the type of cancer, type and amount of peptide, related composition, or combination composition being delivered to the host.

The methods of the invention can be used to treat a variety of cancers. Forms of cancer that may be treated by the delivery or administration of a peptide or analog, derivative or mimetic of this invention and combination therapies containing the same include squamous cell carcinoma, leukemia, acute lymphocytic leukemia, acute lymphoblastic leukemia, B-cell lymphoma, T-cell lymphoma, Hodgkins lymphoma, non-Hodgkins lymphoma, hairy cell lymphoma, Burketts lymphoma, acute or chronic myelogenous leukemias, promyelocytic leukemia, fibrosarcoma, rhabdomyoscarcoma, melanoma, seminoma, teratocarcinoma, neuroblastoma, glioma, astrocytoma, neuroblastoma, glioma, schwannomas; fibrosarcoma, rhabdomyoscaroma, osteosarcoma, melanoma, xeroderma pigmentosum, keratoacanthoma, seminoma, thyroid follicular cancer, and teratocarcinoma. The compositions of this invention also can be useful in the treatment of other carcinomas of the bladder, breast, colon, kidney, liver, lung, ovary, prostate, pancreas, stomach, cervix, thyroid or skin. Compositions of this invention also may be useful in treatment of other hematopoietic tumors of lymphoid lineage, other hematopoietic tumors of myeloid lineage, other tumors of mesenchymal origin, other tumors of the central or peripheral nervous system, and/or other tumors of mesenchymal origin. Advantageously, the methods of the invention also may be useful in reducing cancer progression in prostate cancer cells, melanoma cells (e.g., cutaneous melanoma cells, ocular melanoma cells, and/or lymph node-associated melanoma cells), breast cancer cells, colon cancer cells, and lung cancer cells. The methods of the invention can be used to treat both tumorigenic and non-tumorigenic cancers (e.g., non-tumor-forming hematopoietic cancers). The methods of the invention are particularly useful in the treatment of epithelial cancers (e.g., carcinomas) and/or colorectal cancers, breast cancers, lung cancers, vaginal cancers, cervical cancers, and/or squamous cell carcinomas (e.g., of the head and neck). Additional potential targets include sarcomas and lymphomas. Additional advantageous targets include solid tumors and/or disseminated tumors (e.g., myeloid and lymphoid tumors, which can be acute or chronic).

The present invention also provides methods for enhancing anti-cancer or anti-tumor immunity, comprising administering to a subject in need thereof an effective amount of an isolated VISTA antagonist or a composition containing said isolated VISTA antagonist.

In addition to cancer treatment, the present invention also features a method of treating a pathogen infection, i.e., a bacterial, viral, parasitic or fungal infection, in a subject or host. This method involves administering or otherwise delivering an effective amount of a peptide or analog, derivative or mimetic of this invention so as to reduce the severity, spread, symptoms, or duration of such infection. Such pathogen infections include, but are not limited to diseases caused by bacteria, protozoa, fungi, parasites, or viruses.

In particular embodiments, a viral infection is treated. Any virus normally associated with the activity of effector lymphocytes can be treated by the method. For example, such a method can be used to treat infection by one or more viruses selected from hepatitis type A, hepatitis type B, hepatitis type C, influenza, varicella, adenovirus, herpes simplex type I (HSV-1), herpes simplex type 2 (HSV-2), rinderpest, rhinovirus, echovirus, rotavirus, respiratory syncytial virus, papilloma virus, papilloma virus, cytomegalovirus (CMV, e.g., HCMV), echinovirus, arbovirus, huntavirus, coxsackie virus, mumps virus, measles virus, rubella virus, polio virus, and/or human immunodeficiency virus type I or type 2 (HIV-1, HIV-2). The practice of such methods may result in a reduction in the titer of virus (viral load), reduction of the number of virally infected cells, etc.

In addition to pathogen infections, a peptide or analog, derivative or mimetic of this invention can be administered or otherwise delivered to a subject in association with the treatment of immunoproliferative diseases, immunodeficiency diseases, autoimmune diseases, inflammatory responses, and/or allergic responses.

Moreover, the invention also provides methods for blocking, inhibiting or neutralizing VISTA-mediated T cell suppression and/or stimulating an immune response in a subject, comprising administering to the subject in need thereof an effective amount of an isolated VISTA antagonist or a composition containing said isolated VISTA antagonist. Such methods may be useful for treating a subject with a one or more of a bacterial, viral, parasitic and fungal infections and/or cancer.

### EXAMPLES

The invention now being generally described, it will be more readily understood by reference to the following examples, which are included merely for purposes of illustration of certain aspects and embodiments of the present invention, and are not intended to limit the invention.

### Materials and Methods

Mice

C57BL/6 mice were purchased from Charles River Laboratories. VISTA KO mice on a fully backcrossed C57BL/6 background were obtained from the Mutant Mouse Regional Resource Centers (MMRRC, University of California-Davis, Davis, California, USA) (36). 2D2 TCR transgenic mice were purchased from the Jackson Laboratory (Bar Harbor, ME). PD-1 KO mice were provided by Dr. Honjo (10). PD-L1 KO mice were as described (15). All animals were maintained in a pathogen-free facility at the Medical College of Wisconsin (Milwaukee, WI). All animal protocols were approved by the Institutional Animal Care and Use Committee of the Medical College of Wisconsin.

Mice Necropsy and Semi-Quantitative Pathological Analysis

Age- and sex-matched WT and VISTA KO mice were sacrificed by CO₂ asphyxiation. Organs were harvested, fixed in 10% buffered formalin. H&E stain was performed on tissue sections. Tissue inflammatory status was scored in a blind manner by a pathologist using the following semi-quantitative scoring criteria: 0=normal; 1=mild/small foci of dense lymphocytic infiltrate; 2=moderate/multiple foci of dense/large activated lymphocytic infiltrate with/without germinal center; 3=marked reactive/activated or atypical lymphocytic infiltrate.

Flow Cytometry and Data Analysis

Flow cytometry analysis was performed either on FACScalibur or LSRII using CellQuest software (BD Bioscience, San Jose, CA). Data analyses were performed using FlowJo software (Treestar, Ashland, OR).

Graphs and Statistical Analysis

All graphs and statistical analysis were generated using Prism 4 (GraphPad Software, Inc., CA). Student's t test (two tailed) or two-way ANOVA was used for data analyses. ***P<0.005, **P<0.025, *P<0.05.

### Example 1: Enhancement of T Cell Proliferation

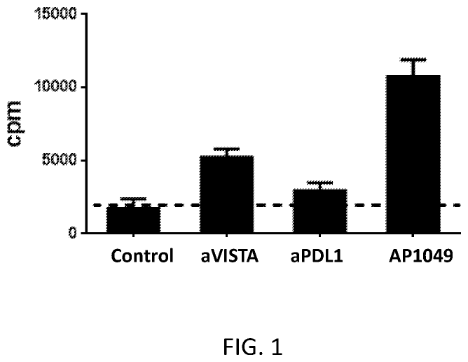

VISTA⁺CD11b⁺ monocytes were enriched from naïve splenocytes using CD11b magnetic beads (Miltenyi). VISTA⁺CD11b^{hi} MHCII⁺ myeloid APCs were FACS sorted, irradiated (2500 rads), and used as antigen-presenting cells to stimulate OT-II transgenic CD4⁺ T cells in the presence of OVA peptide. Control-Ig, monoclonal antibody specific for VISTA and PD-L1 (30 μg/mL), or VISTA-specific peptide (100 μg/mL) were added as indicated. Cell proliferation was measured by tritium incorporation during the last 8 hours of a 72-hour assay. This analysis indicated that T cell proliferation was enhanced in the presence of VISTA or PD-L1 neutralizing monoclonal antibodies, or the AP1049 peptide (FIG. 1). In fact, the AP1049 peptide stimulated T cell proliferation much better than either of the monoclonal antibodies, indicating that the peptide possesses strong antagonistic activity against VISTA.

### Example 2: Enhancement of T Cell Proliferation

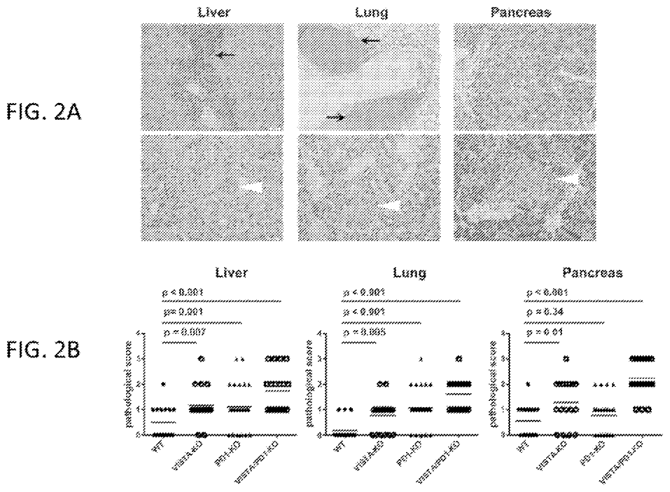

This example relates to the experiment in FIG. 2. Histological analysis of aged VISTA KO, PD-1 KO, and VISTA/PD-1 double KO mice. Necropsy was performed on 12 months old WT (n=16), VISTA KO (n=15), PD-1 KO (n=28), and VISTA/PD-1 double KO (n=25) mice. Organs were fixed, paraffin embedded, sectioned, and stained with H&E. Two representative H&E sections from lung, liver, and pancreas of the VISTA/PD-1 double KO mice were shown in (A). Clusters of tissue-infiltrating leukocytes were marked with black arrows. (Top row) Areas of necrotic tissues were marked with white arrows (Bottom row). All images are of 200× magnification. Scale bar: 50 microns. The inflammatory state of the tissues was evaluated based on a semi-quantitative method that scores the level of the leukocyte infiltration and tissue necrosis (B).

### Example 3: Spontaneous T Cell Activation in the VISTA KO, PD-1 KO, and VISTA/PD-1 Double KO Mice

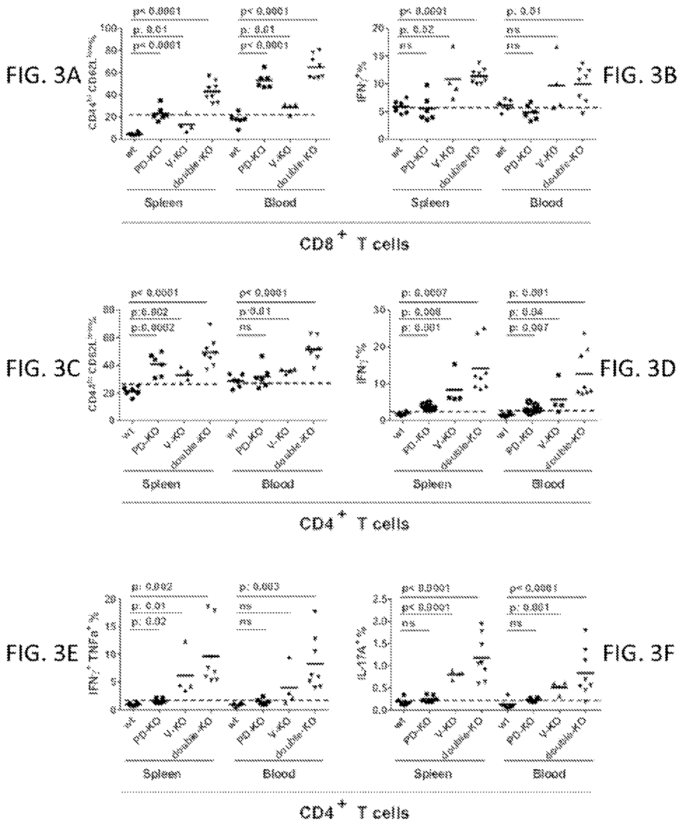

This example relates to the experiments in FIG. 3 showing spontaneous T cell activation in the VISTA KO, PD-1 KO, and VISTA/PD-1 double KO mice. Splenic T cells were collected from age and gender-matched 6-7 months old WT (n=6), VISTA KO (n=4), PD-1 KO (n=6), and VISTA/PD-1 double KO (n=8) mice. The percentages of CD8⁺ and CD4⁺ T cells with activated phenotype (CD44^{hi} CD62L^{lo}) were quantified by flow cytometry. T cells were stimulated ex vivo overnight with soluble anti-CD3/CD28 mAbs, and their cytokine production (i.e. IFNγ, TNFαβγ and IL-17A) was examined by intracellular staining. CD8⁺ T cell phenotypes were shown in A and B. CD4⁺ T cell phenotypes were shown in C-F. Representative results of at least three independent experiments were shown.

### Example 4: Combined Genetic Deficiency of VISTA and PD-1 Exacerbates Autoimmune Disease on the Susceptible Background

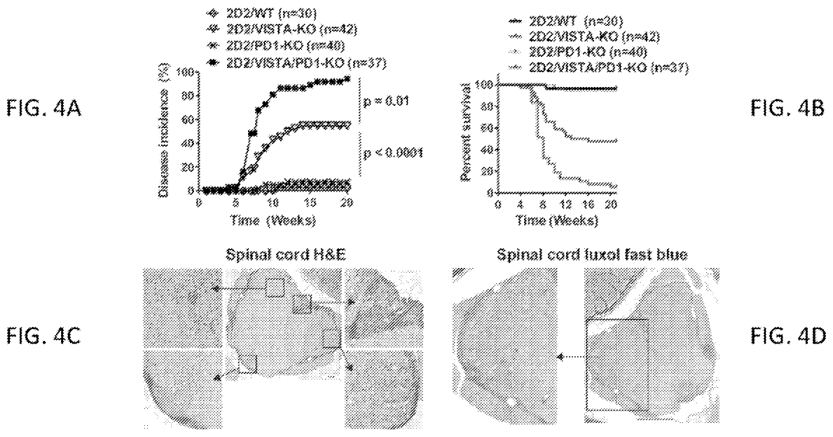

This example relates to the experiments of FIG. 4 showing combined genetic deficiency of VISTA and PD-1 exacerbated autoimmune disease on the susceptible background. The CNS disease incidence (A) and mortality (B) were monitored in 2D2 TCR transgenic mice that were bred onto the VISTA KO, PD-1 KO, and the double KO genetic background. Representative H&E stained spinal cord section from paralyzed double KO mice was shown (C). Enlarged images show areas of extensive lymphocyte infiltration. Luxol fast blue staining of spinal cord sections confirmed extensive demyelination (D). 2D2-WT (n=30), 2D2-VISTA KO (n=42), 2D2-PD-1 KO (n=40), 2D2-VISTA/PD-1 double KO (n=37). Only one 2D2-WT mouse developed disease.

### Example 5: VISTA and the PD-1 Collaboratively Controlled Antigen-Specific T Cell Responses

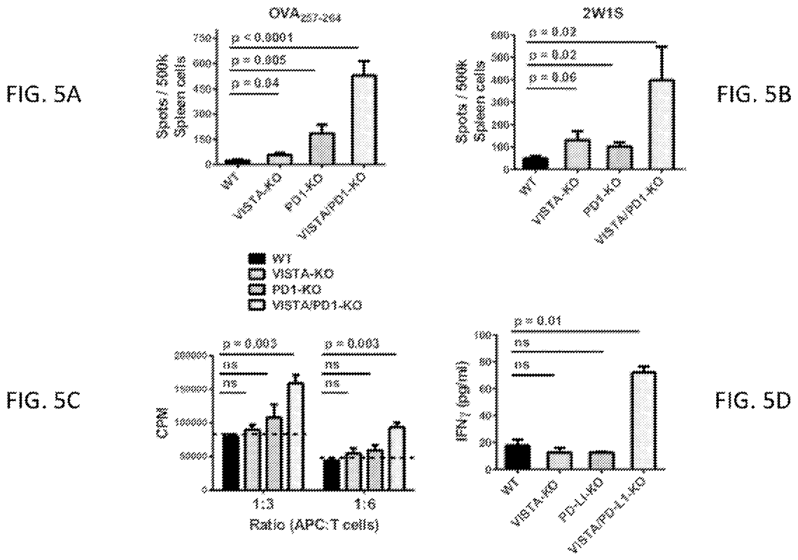

This example relates to FIG. 5 which relates to experiments wherein 6-7 weeks old WT (n=8), VISTA KO (n=9), PD-1 KO (n=7), and VISTA/PD-1 double KO (n=6) mice were immunized with 50 μg soluble peptides OVA²⁵⁷⁻²⁶⁴ (A) or 2W1S (B) together with TLR3 agonist poly (I:C) (100 μg) as adjuvant. Splenocytes were harvested on Day +7 post immunization and re-stimulated with the respective peptides. IFNγ-producing cells were enumerated by the ELISPot assay. To stimulate T cells in vitro, CD11b⁺ CD11c⁺ DCs were sorted from WT, VISTA KO, PD-L1 KO, and VISTA/PD-L1 double KO mice, and incubated with naïve CD4⁺ OTII TCR transgenic T cells in the presence of cognate peptides OVA₃₂₃₋₃₃₉ (10 ng/mL). [³H]-Thymidine was added to the culture for the last 8 hrs of the 72 hrs culture period for measuring T cell proliferation (C). The production of IFNγ was quantified from the culture supernatants by ELISA (D).

### Example 6: Engagement of Both VISTA and PD-L1 During TCR Activation Maximally Suppressed TCR Signaling

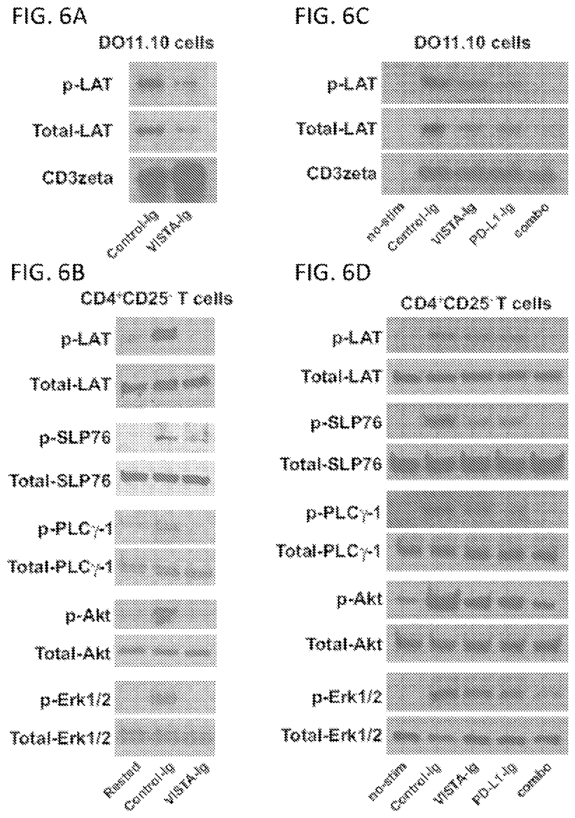

This example relates to the experiments in FIG. 6. Therein it was determined whether VISTA engagement impairs the recruitment of signaling adaptor protein LAT, DO11.10 hybridoma cells (100×10⁶) were stimulated with plate-bound anti-CD3 mAb (2C11, 3 μg/ml), together with co-immobilized control-Ig (8 μg/ml) or VISTA-Ig fusion protein (8 μg/ml) for 10 min at 37° C., and lysed in situ. After removing the unbound cell lysates, plate-bound protein was eluted off the plate, and examined by Western blotting (A). To examine the effect of VISTA on the phosphorylation of TCR signaling molecules, CD25-CD4⁺ T cells were purified from naïve splenocytes and stimulated with plate-bound 2C11 (3 μg/ml) together with control-Ig (8 μg/ml) or VISTA-Ig (8 μg/ml) for 5 min at 37° C. Total cell lysates were prepared and the phosphorylation status of LAT, SLP76, PLC-β1, Akt, and Erk1/2 was examined (B). To determine whether co-engagement of both VISTA and PD-L1 maximally suppresses LAT activation, DO11.10 cells were stimulated with plate-bound 2C11 (2.5p/ml), together with control-Ig (10 μg/ml), or VISTA-Ig (5 μg/ml), or PD-L1-Ig (5 μg/ml), or both Ig fusion proteins. Cells were lysed after 10 min stimulation, and plate-bound proteins were recovered and examined as described above (C). To determine the synergistic effects of engaging both VISTA and PD-L1, pre-activated splenic CD4⁺ T cells were stimulated with plate-bound 2C11 (2.5 μg/ml) together with control-Ig (9 μg/ml), VISTA-Ig (3 μg/ml), PD-L1-Ig (6 μg/ml), or both Ig fusion proteins for 10 min at 37° C. Total cell lysates were harvested for Western blotting analysis (D). Representative results from 2-3 independent experiments were shown.

### Analysis

Immunogenic bladder carcinoma tumors (MB49) were inoculated in female mice. AP1049 was tested for its ability to slow tumor growth and/or facilitate tumor regression. The readout for this assay was tumor growth.

MB49 tumors were inoculated in female mice (300k) via intradermal (i.d.) inoculation, which facilitates measurement of tumor size. Mice were treated with either PBS (control) or VISTA antagonist peptide (AP1049), via daily injections around tumor mass starting on day +1 and continuing for 2 weeks. Tumor size was measured by caliper every 2-3 days.

Using these methods slowed tumor growth and/or tumor regression in mice treated with AP1049 was obtained as compared with mice treated with control.

As shown in FIG. 3, AP1049 treatment reduced tumor growth in the MB49 tumor model, indicating that the peptide may bind to the critical/active site of VISTA and block the immune-suppressive function of VISTA.

### CONCLUSIONS

Loss of T-Cell Peripheral Tolerance Upon Combined Genetic Disruption of VISTA and PD-1, or PD-L1

In order to determine whether VISTA and PD-1 regulate immune responses in a redundant or independent/synergistic manner, Vista^{-/-}Pdcd1^{-/-} mice (herein referred to as VISTA/PD-1 double KO) were generated on the C57BL/6 background and characterized. The double KO mice were born fertile and produced normal litter sizes. Normal thymic development and lymphocyte populations (T, B, NK, and NKT cells) in the bone marrow, spleen, and lymph nodes were observed in 6-8 week old KO mice.

Comprehensive multi-organ histological analyses were performed in 12 months old WT, VISTA KO, PD-1 KO, and the VISTA/PD-1 double KO mice (FIG. 2). Hematoxylin and eosin (H&E) stained sections from heart, lung, liver, kidney, pancreas, salivary gland, small and large intestines, and brain were examined. Several organs, including lung, liver and pancreas in the double KO mice were heavily infiltrated with leukocytes (FIG. 2A, top row), and showed significant tissue necrosis, presumably due to immune cell-mediated destruction (FIG. 1A, bottom row). The levels of leukocyte infiltration and tissue necrosis in the KO mice were blindly quantified based on a semi-quantitative scoring method, and the VISTA/PD-1 double KO mice showed the highest scores when compared to WT and single KO mice (FIG. 2B). Despite the significant accumulation of activated T cells, the double KO mice did not develop overt autoimmune disease. Serum levels of IgM and IgA were moderately elevated in aged double KO mice. Our co-housed PD-1 KO mice also developed accumulation of spontaneously activated T cells and chronic inflammation in multiple organs, but failed to develop arthritis (20× mice were analyzed at the age of 12 months), or other previously reported autoimmune phenotypes (10). This discrepancy might be due to the different housing conditions or the age of the mice analyzed.

When compared with VISTA KO and PD-1 KO mice, VISTA/PD-1 double KO mice at the age of 6-7 months showed significantly increased frequencies of CD44^{hi}CD62L^{lo} CD8⁺ and CD4⁺ T cells, which is indicative of an activated or memory phenotype (FIGS. 3A and C). Upon in vitro re-stimulation, the double KO T cells produced significantly higher levels of cytokines, such as IFNγ, TNFα, and IL-17A than WT and single KO cells (FIG. 3B, D-F).

PD-1 binds to ligands PD-L1 and PD-L2 (20). To corroborate the results seen in the VISTA/PD-1 double KO mice, we bred VISTA KO onto the previously described PD-L1 KO (15), and generated the VISTA/PD-L1 double KO mice. Our data demonstrated spontaneous activation of peripheral CD4⁺ and CD8⁺ T cells in the VISTA/PD-L1 double KO mice, which was comparable to that of VISTA/PD-1 double KO mice (not shown). Together, these data support the conclusion that VISTA and the PD-1/PD-L1 pathway non-redundantly control the peripheral tolerance of T cells.

The phenotype of spontaneous T cell activation in the double KO mice (FIG. 3) indicates that both VISTA and PD-1 regulate the threshold of TCR activation to auto-antigens. We hypothesize that disruption of both pathways will increase predisposition to autoimmune disease on a susceptible background. To test this hypothesis, the VISTA/PD-1 double KO mice were bred with the 2D2 mice, which express a TCR transgene specific for the self-antigen, myelin oligodendrocyte glycoprotein (MOG₃₅₋₅₅) (21). Previous studies reported that 4% of 2D2 mice developed spontaneous EAE between the age of 3-5 months (21). A similar incidence of spontaneous EAE (1/30, ˜3%) was observed in our colony of 2D2-WT mice under 6 months of age (FIG. 4A1). 2D2-PD-1 KO mice showed similar incidence of spontaneous disease as the WT mice (2/40, 5%). In contrast, genetic deficiency of VISTA accelerated disease onset, such that ˜60% (25/42) of the 2D2-VISTA KO mice rapidly succumbed to complete hind limb paralysis within 3 months. Combined deficiency of VISTA and PD-1 further increased disease incidence to ˜90% (35/37) (FIGS. 4A and B). Analysis of CNS tissue from paralyzed 2D2-VISTA/PD-1 double KO mice confirmed the accumulation of inflammatory cells and demyelination (FIGS. 4C and D). The disease onset age ranged between 4-16 weeks in the 2D2-VISTA/PD-1 double KO mice, which was similar to those observed in the 2D2-VISTA KO mice (5-16 weeks) (not shown). Only one WT mouse developed disease around 12 weeks of age. A small percentage of diseased mice from the 2D2-VISTA KO (2/40) and 2D2-VISTA/PD-1 double KO (3/37) mice developed atypical EAE, manifested as unilateral paralysis rather than bi-lateral paralysis.

Combined Disruption of VISTA and PD-1 Synergistically Augments T Cell Responses Upon Antigen Challenge

Spontaneous T cell activation and enhanced autoimmunity in aged VISTA/PD-1 double KO mice indicate that VISTA and PD-1 control T cell tolerance towards auto-antigens. We hypothesize that these pathways also critically regulate T cell responses towards foreign antigens. To address this question, mice were immunized with soluble antigenic peptides together with poly (I:C) (TLR3 agonist) as adjuvant. 2W1S, an MHC class II-restricted peptide, and OVA²⁵⁷⁻²⁶⁴ an MHC class I-restricted peptide were used (22). On day +7 post-immunization, splenic T cells were isolated and restimulated ex vivo with the respective peptides. Significantly higher numbers of IFNγ-producing T cells were present in the VISTA/PD-1 double KO mice, compared to WT or single KO mice, indicating that VISTA and PD-1 non-redundantly control T cell responses (FIG. 5A-B).

We have previously reported that VISTA functions as a ligand that engages an unknown receptor on T cells and suppresses T cell activation (3). Since both VISTA and PD-L1 are highly expressed on CD11b+ myeloid APCs (2, 3), we hypothesize that a combined deficiency of VISTA and PD-L1 on APCs maximally enhances T cell activation. To test this hypothesis, CD11b+ myeloid DCs were isolated from WT, VISTA KO, PD-L1 KO, and the VISTA/PD-L1 double KO mice (15, 23), and used to stimulate naïve CD4⁺ OTII TCR transgenic T cells, in the presence of cognate peptide OVA₃₂₃₋₃₃₉. Our data show that the combined deficiency of VISTA and PD-L1 on myeloid APCs synergistically enhanced T cell proliferation and IFNγ production (FIGS. 5C and D).

Although the receptor for VISTA (VISTA-R) is unknown, we speculate that the engagement of VISTA-R on T cells suppresses TCR signaling independent of PD-1. Additionally, we hypothesize that the co-engagement of VISTA-R and PD-1 on T cells synergistically impairs TCR signaling. To test these hypotheses, proximal TCR signaling events were examined using immobilized fusion proteins VISTA-Ig and PD-L1-Ig, both of which suppressed T cell proliferation and cytokine production in vitro (3, 12). LAT is a proximal signaling adaptor that is phosphorylated upon TCR stimulation, and forms a complex with multiple signaling molecules including SLP76 and PLC-γ1 (24). To determine whether VISTA functions by interfering with the phosphorylation of LAT, a solid phase immunoprecipitation assay was performed, where the proximal signaling complexes could be recovered as the bound fraction to the plastic surface (25, 26). Our data show that in the presence of co-immobilized control-Ig or VISTA-Ig proteins, plate-bound anti-CD3ε mAb pulled-down comparable amounts of CD3ζ. This result excluded the possibility that the immobilized VISTA-Ig displaced anti-CD38 mAb or impaired the binding of anti-CD38 mAb to the TCR/CD3 complex (FIG. 6A). Despite the similar engagement of CD3C, immobilized VISTA-Ig significantly reduced the amount of LAT recruited to the CD3 complex, and its phosphorylation (FIG. 6A). When total cell lysates were examined, the phosphorylation of several proximal and downstream signaling molecules, such as SLP76, PLC-γ1, Akt, and Erk1/2 were also impaired (FIG. 6).

Next, the effect of co-engaging VISTA-Ig and PD-L1-Ig was examined (FIGS. 6C and D). Pre-activated T cells were analyzed, as they expressed high level of PD-1 (27). Consistent with our hypothesis, the combined engagement of VISTA-Ig and PD-L1-Ig maximally reduced the phosphorylation of LAT and its recruitment to the CD3 complex (FIG. 5C). Furthermore, VISTA-Ig and PD-L1-Ig co-engagement maximally reduced the phosphorylation of SLP76, PLC-γ1, Akt, and Erk1/2 in total cell lysates (FIG. 6D). Together, these results support the conclusion that VISTA-R and PD-1 each impairs early TCR signaling, and results in the most robust suppression when combined.

### Discussion

VISTA and PD-1 both function as immune checkpoint proteins that suppress T cell activation. They share overlapping expression patterns within the hematopoietic compartment. It is therefore important to define their independent immune-regulatory roles.

Evidence based on studies of the KO mice indicates that VISTA and PD-1 non-redundantly regulate immune responses. Genetic disruption of VISTA accelerated autoimmune disease on a susceptible background, as well as resulted in multi-organ chronic inflammation due to spontaneous T cell activation (22). Aged PD-1 KO mice were reported to develop late onset autoimmunity in the C57BL/6 background (10). Our study of PD-1 KO mice also showed accumulation of spontaneously activated T cells and chronic inflammation in multiple organs. Furthermore, in the current study of the VISTA/PD-1 double KO mice, we provided strong evidence for independent control of T cell responses by these two checkpoints. Synergistic or additive T cell activation was observed from aged double KO mice when compared to the single KO mice, which might reflect the lack of “brakes” in TCR signaling, resulting in lower threshold of T cell activation and loss of peripheral tolerance to auto-antigens. Similarly, synergistic T cell responses were observed in double KO mice in response to foreign antigens.

We hypothesize that VISTA and PD-1 both function as brakes for T cell activation. Since APCs lacking both VISTA and PD-L1 stimulated T cells better than single KO APCs or WT APCs in vitro, this data indicates that VISTA and PD-L1 engage independent inhibitory receptors on T cells. To determine the effects of VISTA-R and PD-1 on TCR signaling, immobilized VISTA-Ig and PD-L1-Ig were used to engage VISTA-R and PD-1, respectively. Our data shows that VISTA-Ig and PD-L1-Ig fusion proteins impaired the activation of LAT, as well as the phosphorylation of proximal signaling molecules (SLP76 and PLC-yl) and downstream molecules (Akt and Erk1/2). The co-engagement of both Ig fusion proteins to their receptors resulted in additive effects. Based on these data, we conclude that similar to PD-1, VISTA-R impairs early TCR signaling. PD-1 has been shown to accumulate at the immune synapse and recruits SHP-1/2 to downregulate TCR signaling (33, 34). Whether or not similar phosphatases might be involved in VISTA-R-mediated effects remains to be determined.

We speculate that multiple mechanisms underlie the synergistic T cell activation when both VISTA and PD-1 are blocked in vivo. In addition to being a ligand, VISTA acts as a receptor that transduces inhibitory signals during T cell activation (6). This function likely contributes to the synergistic T cell activation seen in the VISTA/PD-1 double KO mice. Furthermore, VISTA might exert T cell extrinsic functions. VISTA is highly expressed on myeloid cells such as Cd11b⁺ DCs and macrophages (3). Our future studies will dissect lineage-specific roles of VISTA in controlling both innate and adaptive immune responses.

Additional immune regulatory molecules, such as LAG3 and Tim3 have been shown to synergize with PD-1 to control T cell responses (19, 35). Our current study shows that VISTA and PD-1 synergistically regulate T cell responses against self- and foreign-antigens, and concurrently targeting both molecules leads to optimal therapeutic efficacy in murine tumor models. The absence of overt autoimmune disease in the double KO mice suggests that the combinatorial blockade of VISTA and PD-1 might achieve optimal therapeutic efficacy with less severe immune-related adverse events, therefore more amenable for the treatment of cancer.

The following references and all other references which are cited in this application are incorporated by reference in their entireties herein.

### REFERENCES

1. Chen L, Flies D B. Molecular mechanisms of T cell co-stimulation and co-inhibition. Nat Rev Immunol 2013; 13: 227-42.
2. Greenwald R J, Freeman G J, Sharpe A H. The B7 family revisited. Annu Rev Immunol 2005; 23: 515-48.
3. Wang L, Rubinstein R, Lines J L, et al. VISTA, a novel mouse Ig superfamily ligand that negatively regulates T cell responses. J Exp Med 2011; 208: 577-92.
4. Flies D B, Wang S, Xu H, Chen L. Cutting edge: A monoclonal antibody specific for the programmed death-1 homolog prevents graft-versus-host disease in mouse models. J Immunol 2011; 187: 1537-41.
5. Lines J L, Pantazi E, Mak J, et al. VISTA Is an Immune Checkpoint Molecule for Human T Cells. Cancer Res 2014; 74: 1924-32.
6. Flies D B, Han X, Higuchi T, et al. Coinhibitory receptor PD-1H preferentially suppresses CD4+ T cell-mediated immunity. J Clin Invest 2014.
7. Le Mercier I, Chen W, Lines J L, et al. VISTA Regulates the Development of Protective Antitumor Immunity. Cancer Res 2014; 74: 1933-44.
8. Tivol E A, Borriello F, Schweitzer A N, Lynch W P, Bluestone J A, Sharpe A H. Loss of CTLA-4 leads to massive lymphoproliferation and fatal multiorgan tissue destruction, revealing a critical negative regulatory role of CTLA-4. Immunity 1995; 3: 541-7.
9. Waterhouse P, Penninger J M, Timms E, et al. Lymphoproliferative disorders with early lethality in mice deficient in Ctla-4. Science 1995; 270: 985-8.
10. Nishimura H, Nose M, Hiai H, Minato N, Honjo T. Development of lupus-like autoimmune diseases by disruption of the PD-1 gene encoding an ITIM motif-carrying immunoreceptor. Immunity 1999; 11: 141-51.
11. Nishimura H, Okazaki T, Tanaka Y, et al. Autoimmune dilated cardiomyopathy in PD-1 receptor-deficient mice. Science 2001; 291: 319-22.
12. Freeman G J, Long A J, Iwai Y, et al. Engagement of the PD-1 immunoinhibitory receptor by a novel B7 family member leads to negative regulation of lymphocyte activation. J Exp Med 2000; 192: 1027-34.
13. Latchman Y, Wood C R, Chernova T, et al. PD-L2 is a second ligand for PD-1 and inhibits T cell activation. Nat Immunol 2001; 2: 261-8.
14. Keir M E, Liang S C, Guleria I, et al. Tissue expression of PD-L1 mediates peripheral T cell tolerance. J Exp Med 2006; 203: 883-95.
15. Guleria I, Khosroshahi A, Ansari M J, et al. A critical role for the programmed death ligand 1 in fetomaternal tolerance. J Exp Med 2005; 202: 231-7.
16. Zhu G, Augustine M M, Azuma T, et al. B7-H4-deficient mice display augmented neutrophil-mediated innate immunity. Blood 2009; 113: 1759-67.
17. Okazaki T, Okazaki I M, Wang J, et al. PD-1 and LAG-3 inhibitory co-receptors act synergistically to prevent autoimmunity in mice. J Exp Med 2011; 208: 395-407.
18. Jin H T, Anderson A C, Tan W G, et al. Cooperation of Tim-3 and PD-1 in CD8 T-cell exhaustion during chronic viral infection. Proc Natl Acad Sci USA 2010; 107: 14733-8.
19. Sakuishi K, Apetoh L, Sullivan J M, Blazar B R, Kuchroo V K, Anderson A C. Targeting Tim-3 and PD-1 pathways to reverse T cell exhaustion and restore anti-tumor immunity. J Exp Med 2010; 207: 2187-94.
20. Keir M E, Butte M J, Freeman G J, Sharpe A H. PD-1 and its ligands in tolerance and immunity. Annu Rev Immunol 2008; 26: 677-704.
21. Bettelli E, Pagany M, Weiner H L, Linington C, Sobel R A, Kuchroo V K. Myelin oligodendrocyte glycoprotein-specific T cell receptor transgenic mice develop spontaneous autoimmune optic neuritis. J Exp Med 2003; 197: 1073-81.
22. Wang L, Le Mercier I, Putra J, et al. Disruption of the immune-checkpoint VISTA gene imparts a proinflammatory phenotype with predisposition to the development of autoimmunity. Proc Natl Acad Sci USA 2014; 111: 14846-51.
23. Wang L, Pino-Lagos K, de Vries V C, Guleria I, Sayegh M H, Noelle R J. Programmed death 1 ligand signaling regulates the generation of adaptive Foxp3+CD4+ regulatory T cells. Proc Natl Acad Sci USA 2008; 105: 9331-6.
24. Dustin M L, Depoil D. New insights into the T cell synapse from single molecule techniques. Nat Rev Immunol 2011; 11: 672-84.
25. Lysechko T L, Ostergaard H L. Differential Src family kinase activity requirements for CD3 zeta phosphorylation/ZAP70 recruitment and CD3 epsilon phosphorylation. J Immunol 2005; 174: 7807-14.
26. Muhammad A, Schiller H B, Forster F, et al. Sequential cooperation of CD2 and CD48 in the buildup of the early TCR signalosome. J Immunol 2009; 182: 7672-80.
27. Pentcheva-Hoang T, Chen L, Pardoll D M, Allison J P. Programmed death-1 concentration at the immunological synapse is determined by ligand affinity and availability. Proc Natl Acad Sci USA 2007; 104: 17765-70.
28. Leach D R, Krummel M F, Allison J P. Enhancement of antitumor immunity by CTLA-4 blockade. Science 1996; 271: 1734-6.
29. Holmgaard R B, Zamarin D, Munn D H, Wolchok J D, Allison J P. Indoleamine 2,3-dioxygenase is a critical resistance mechanism in antitumor T cell immunotherapy targeting CTLA-4. J Exp Med 2013; 210: 1389-402.
30. Spranger S, Koblish H K, Horton B, Scherle P A, Newton R, Gajewski T F. Mechanism of tumor rejection with doublets of CTLA-4, PD-1/PD-L1, or IDO blockade involves restored IL-2 production and proliferation of CD8(+) T cells directly within the tumor microenvironment. J Immunother Cancer 2014; 2: 3.
31. Curran M A, Montalvo W, Yagita H, Allison J P. PD-1 and CTLA-4 combination blockade expands infiltrating T cells and reduces regulatory T and myeloid cells within B16 melanoma tumors. Proc Natl Acad Sci USA 2010; 107: 4275-80.
32. Klebanoff C A, Khong H T, Antony P A, Palmer D C, Restifo N P. Sinks, suppressors and antigen presenters: how lymphodepletion enhances T cell-mediated tumor immunotherapy. Trends Immunol 2005; 26: 111-7.
33. Sheppard K A, Fitz L J, Lee J M, et al. PD-1 inhibits T-cell receptor induced phosphorylation of the ZAP70/CD3zeta signalosome and downstream signaling to PKCtheta. FEBS Lett 2004; 574: 37-41.
34. Chemnitz J M, Parry R V, Nichols K E, June C H, Riley J L. SHP-1 and SHP-2 associate with immunoreceptor tyrosine-based switch motif of programmed death 1 upon primary human T cell stimulation, but only receptor ligation prevents T cell activation. J Immunol 2004; 173: 945-54.
35. Woo S R, Turnis M E, Goldberg M V, et al. Immune inhibitory molecules LAG-3 and PD-1 synergistically regulate T-cell function to promote tumoral immune escape. Cancer Res 2011; 72: 917-27.
36. Tang T, Li L, Tang J, et al. A mouse knockout library for secreted and transmembrane proteins. Nat Biotechnol 2010; 28: 749-55.

What is claimed is:

1-40. (canceled)

41. A method of eliciting a synergistic or additive effect on T cell activation, CD4+ T cells, CD8+ T cells, or TH1 or CTL immunity comprising administering
(i) a synergistically or additively effective amount of (a) an antibody or polypeptide that specifically binds to and antagonizes VISTA and (b) an antibody or polypeptide that specifically binds to and antagonizes PD-1, PD-L1, or a PD-L2 polypeptide; or
(ii) a synergistically or additively effective amount of (i) a VISTA agonist selected from an antibody that specifically binds and agonizes VISTA or a VISTA-Ig fusion protein and (ii) a PD-1 agonist comprising an antibody or antibody fragment that specifically binds and agonizes PD-1, or a PD-1, PD-L1 or PD-L2 Ig fusion protein.

42. The method of claim 41, wherein the Ig of the antibody or in the Ig fusion polypeptide is an IgG1, IgG2, IgG3 or IgG4 or chimera or fragment thereof.

43. The method of claim 41, wherein the Ig of the antibody or the Ig fusion protein is an IgG1, IgG2, IgG3 or IgG4 or chimera or fragment thereof.

44. The method of claim 41, wherein the agonistic anti-VISTA antibody and/or the agonistic anti-PD-1 antibody the antibody comprises human IgG1, IgG2, IgG3 or IgG4 constant domains.

45. A method of eliciting a synergistic or additive effect on anti-tumor immunity according to claim 41 (i), comprising administering a synergistically or additively effective amount of (i) an antagonistic antibody that specifically binds VISTA and (ii) an antagonistic antibody that specifically binds PD-1.

46. A method of eliciting a synergistic or additive effect on inhibiting autoimmunity, allergy or inflammation according to claim 41 (ii), comprising administering a synergistically or additively effective amount of (i) a VISTA agonist selected from an agonistic antibody that specifically binds VISTA or a VISTA-Ig fusion protein and (ii) a PD-1 agonist selected from an agonistic antibody that specifically binds PD-1 or a PD-1-Ig, PD-L1-Ig, or PD-L2-Ig fusion protein.

47. The method of claim 45, wherein the combination has a synergistic or additive effect on tumor growth, tumor cell invasion, or metastasis.

48. The method of claim 46, wherein the agonistic antibody that specifically binds VISTA and/or agonistic antibody that specifically binds PD-1 is a human, chimeric or humanized antibody or antibody fragment.

49. The method of claim 48, wherein the agonistic antibody that specifically binds VISTA and/or agonistic antibody that specifically binds PD-1 comprises human IgG1, IgG2, IgG3 or IgG4 constant domains.

50. The method of claim 45, which is used to treat a cancer selected from leukemia, lymphoma, lung cancer, melanoma, sarcoma, ovarian cancer, breast cancer, brain cancer, or any other solid tumor.

51. The method of claim 49 which is used to treat allergy, autoimmunity or inflammation.

52. A method of eliciting a synergistic or additive effect on immunity against an infectious agent or infected cells according to claim 41, comprising administering a synergistically or additively effective amount of (i) an antibody that specifically binds VISTA and (ii) an antibody that specifically binds PD-1.

53. The method of claim 52, wherein the combination has a synergistic or additive effect on the inhibition of proliferation or growth, invasion of host cells by the infectious agent, or CTL, CD4+ or CD8+ killing of infected cells.

55. A composition comprising
(i) the combination of a VISTA antagonist comprising an antibody or antibody fragment that specifically binds to and antagonizes VISTA and a PD-1 antagonist selected from an antibody or antibody fragment that binds to PD-1, PD-L1 or PD-L2, which combination elicits a synergistic or additive effect on promoting T cell immunity; or
(ii) the combination of a VISTA agonist selected from an antibody or antibody fragment that specifically binds and agonizes VISTA or a VISTA-Ig fusion protein and a PD-1 agonist comprising an antibody or antibody fragment that specifically binds and agonizes PD-1, or a PD-1, PD-L1 or PD-L2 Ig fusion protein, which combination elicits a synergistic or additive effect on inhibiting T cell immunity.


SEQUENCE LISTING (1 sequence)

SEQ ID NO: 1 - protein, 12 aa, synthetic construct
  REGION 1..12: note = Isolated VISTA Antagonist
SSACDWIKRSCH